(19) United States
(12) Patent Application Publication
(10) Pub. No.: US 2003/0064053 A1
(43) Pub. Date: Apr. 3, 2003

(54) Multivalent protein conjugate with multiple ligand-binding domains of receptors

(76) Inventors: Shengjiang Liu, Mountain View, CA (US); Jean-Francois Martini, Redwood City, CA (US); Dayou Liu, Camarillo, CA (US)

Correspondence Address: WILSON SONSINI GOODRICH & ROSATI, 650 PAGE MILL ROAD, PALO ALTO, CA, 943041050

(21) Appl. No.: 10/232,838

(22) Filed: Aug. 30, 2002

Related U.S. Application Data
(60) Provisional application No. 60/316,718, filed on Aug. 31, 2001.

(51) Int. Cl. A61K 38/20; C07K 14/715

(52) U.S. Cl. 424/85.2; 530/350; 530/351

(57) ABSTRACT

The present invention provides compositions and methods for treating abnormal cell proliferation and for regulating angiogenesis. In particular, multivalent protein conjugates (MVPs) are constructed to include multiple ligand-binding domains of different receptors and utilized to target multiple, different ligands that are involved in regulation of cell growth and neovascularization. The MVPs of the present invention can be used to treat various conditions associated with abnormal cell proliferation and angiogenesis such as cancer and cardiovascular disorders, as well as to promote wound healing.

### CROSS REFERENCE TO RELATED APPLICATIONS

This application claims the benefit of U.S. Provisional Application Serial No: 60/316,718 entitled “Multivalent protein conjugate with multiple ligand-binding domains of receptors” filed Aug. 31, 2001. This application is incorporated herein by reference.

### FIELD OF THE INVENTION

1. Field of the Invention

This invention relates to methods and compositions for treating conditions associated with abnormal cell proliferation such as cancer, and with angiogenesis such as tumors, wound healing, and cardiovascular disorders. More particularly, this invention relates to methods for treating these conditions using multivalent protein conjugates which include multiple ligand-binding domains of receptors such as nuclear hormone receptors and receptors for angiogenic factor such as vascular endothelial growth factors (VEGFs), basic fibroblast growth factor (bFGF), angiopoietins (AGP) and angiogenic inhibitors such as thrombospondins (TSP), angiostatin, and endostatin.

2. Description of Related Art

Over the past thirty years, significant advances in the chemotherapy of neoplastic diseases have been realized. Lately biologic agents such as therapeutic antibodies have been approved by the FDA for treatment of cancer.

In general, therapeutic agents currently used in clinical cancer therapy can be categorized into six groups: alkylating agents, antibiotic agents, antimetabolic agents, biologic agents, hormonal agents, and plant-derived agents. Limited successes have been achieved clinically significant advances in the chemotherapy of a number of neoplastic diseases, including choriocarcinoma, Wilm's tumor, acute leukemia, rhabdomyosarcoma, retinoblastoma, Hodgkin's disease and Burkitt's lymphoma. However, for many forms of cancer especially malignant solid tumors, the treatment remains fraught with complications and side effects which often present an array of suboptimal treatment choices.

The most significant underlying problem associated the side effects of chemotherapy is the non-specific killing of fast-dividing cells, including blood cells and hair matrix cells. For therapeutic interventions using chemotherapy certain types of tumors have been more amenable than others to the treatment. For example, the soft tissue tumors (e.g., lymphomas), and tumors of the blood and blood-forming organs (e.g., leukemias) have generally been more responsive to chemotherapeutic therapy than have solid tumors such as carcinomas. One reason for this is the greater physical accessibility of lymphoma and leukemic cells to chemotherapeutic intervention. However, it is much more difficult for most chemotherapeutic agents to reach all of the cells of a solid tumor mass than it is the soft tumors and blood-based tumors, and therefore much more difficult to achieve a total cell kill. The toxicities associated with most conventional antitumor agents then become the limiting factors.

Over the past 30 years, fundamental advances in our knowledge of the basic science underlying neoplastic processes at the cellular and tissue level have been made. To develop therapeutics more specifically targeting tumors, much research over the years has focused on identifying tumor-specific “marker antigens” that can serve as immunological targets both for chemotherapy and diagnosis. Many tumor-specific, or quasi-tumor-specific (“tumor-associated”), markers have been identified as tumor cell antigens that can be recognized by specific antibodies. Immunotoxins that are conjugates of a specific targeting agent typically a tumor-directed antibody or fragment, with a cytotoxic agent, such as a toxin moiety, have been developed with the hope to selectively kill cells carrying the targeted antigen. Unfortunately, it is generally the case that the so-called tumor specific antibodies in and of themselves do not exert sufficient antitumor effects to make them useful in cancer therapy.

More recently, great interests have been provoked by advances in the knowledge of how tumors grow via neovascularization or angiogenesis. As used herein, angiogenesis means the generation of new blood vessels into a tissue or organ.

Angiogenesis is an important process of developing new blood vessels that involves the proliferation, migration and tissue infiltration of capillary endothelial cells from existing blood vessels. Angiogenesis is involved in both normal physiological processes including embryonic development, follicular growth, and wound healing, and in pathological conditions involving tumor proliferation, metastasis, and non-neoplastic diseases involving abnormal neovascularization in neovascular glaucoma (Folkman, J. and Klagsbrun, M. Science 235:442-447 (1987).

Under normal physiological conditions, humans or animals only undergo angiogenesis in very specific restricted situations. For example, angiogenesis is normally observed in wound healing, fetal and embryonal development and formation of the corpus luteum, endometrium and placenta. The control of angiogenesis is a highly regulated system of angiogenic stimulators and inhibitors. The control of angiogenesis has been found to be altered in certain disease states and, in many cases, the pathological damage associated with the disease is related to the uncontrolled angiogenesis such as that in a malignant solid tumor. It has been recognized that the tumor growth is always accompanied by angiogenesis and solid tumor nodules become dormant at 2-3 mm without neovascularization (Folkman, J. 1971, New. Eng. J. of Med., 18, 1182-1186).

Physiologically, both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis and abnormal growth by endothelial cells and supports the pathological damage seen in these conditions. The diverse pathological states created due to unregulated angiogenesis have been grouped together as angiogenic dependent or angiogenic associated diseases. Therapies directed at control of the angiogenic processes could lead to the abrogation or mitigation of these diseases.

At the molecular level, many growth factors, cytokines, receptor tyrosine kinases, and natural occurring factors are involved at various determinant point of new blood vessel formation (Table I as shown in FIG. 6). Among the growth factors, vascular endothelial growth factors (VEGFs) (Lars Holman, Michael O'Reilly & Judah Folkman, 1995, Nature Medicine, 1,149-152; Dongfang Wang, David Donner, and Robert Warren, 2000, J. Biol. Chem. 275, 15905-15911) and basic fibroblast growth factor (bFGF) (Montesano R. et al, Proc Natl Acad Sci U S A, 83(19):7297-301, 1986) are the prominent ones that play significant roles in angiogenesis.

Other growth factors involved in angiogenesis include angiopoietins (Davis, S. et al, Cell 87,1161-1169, 1996; Isau, W. Nature 386,631-642, 1997; Kim, I. et al Circulation Research 86(9), 952-959, 2000, Valenzuela, David et al; Proc. Natl Acad. Sci USA, 96, 1904-1909, 1999), ephrines (Holder, N. et al, 1999, Development 126,2033-2044), thrombospondins (TSP) (Iruela-Arispe M. et al, 1991, Proc Natl Acad. USA 1991 88,5026-5030, Volpert, O. V. et al, Biochem. Biophys. Res Comm.1995, 217,326-332), neuropilins (NP) (Soker S. et al, 1998, Cell, 92:735-45), Del 1 protein, platlet derived growth factor (PDGF) (Antoniades H. N. et al, 1979, Proc Natl Acad Sci U S A 76(4):1809-13), h-endostatin (hereinafter “endostatin”) (O'reilly, M. et al, 1997,Cell,88:277-285) and h-angiostatin (hereinafter “angiostatin”) (O'reilly, M. et al, 1994, Cell, 79:315-328), angiocidin (Juszynski, G., 2001, 92 AARC, New Orleans, March 25-April 2), placental growth factor (PIGF) (Maglione D. et al., Proc Natl Acad Sci U S A. 1991, 88(20):9267-711993, and Oncogene, 8(4):925-31,1993), tumor necrosis alpha (TNFα (Sopotsinskaia EB et al, Patol Fiziol Eksp Ter (5):62-4, 1988, and Maas J W et al, Fertil Steril 75(1):180-5,2001). Interactions of these growth factors with their cognate receptors on the cell surface, e.g., bFGF/FGFR, VEGF/VEGFR and Angiopoietin/Tie2 receptor interactions, are thought to be crucial for angiogenesis and vascular remodeling. Under normal physiological conditions, these substances exert their regulatory activity on angiogenesis at a relatively more accurately balanced manner as compared with uncontrolled angiogenesis under pathological conditions.

VEGF-related growth factors are important for tumor angiogenesis (Nicosia R. Amer. J. Pathol. 153;11-16, 1998). So far four types of VEGF have been identified from mammalian tissues including VEGF or VEGF-A (that has several isoforms based on the number of amino acid residuals: 206, 189, 165, 145, and 121), VEGF-B (Olosson et al 1996), VEGF-C (Joukov et al, EMBO J. 15(7):1751 1996, Joukov et al, EMBO J. 15(2):290-98, 1996 and Lee J. et al, Proc. Natl. Acad. Sci. USA, 93:1988-1992, 1996) and VEGF-D (Orlandini et al, Proc. Natl. cad. Sci. USA, 93;11675-11680, 1996 and Achen, M., el al. Proc. Natl. Sci Acad. USA, 95:548-553,1996). A gene encoding a polypeptide with ˜25% amino acid identity to mammalian VEGF was identified in the genome of Orf virus (OV), a parapoxvirus that affects sheep and goats and occasionally, humans, to generate lesions with angiogenesis. The is called VEGF-E (Lyttle D J, etal, J Virol. 68(1):84-92, 1994 and Ogawa, S. et al, J. Biol Chem, 273(47); 31273-31282, 1998).

VEGF-R1 (Flt1) (Shibuya, M. et al, Oncogene 5:519-524, 1990) binds specifically to VEGF-A, VEGF-B, and PIGF. VEGF-R2 (KDR) (Terman B. I. et al, Oncogene 6:1677-1683, 1991) binds to VEGF-A, VEGF-C and VEGF-D. The third receptor (Flt4) binds to VEGF-C and VEGF-D. Interactions between VEGF and Flt1 or KDR result in the vasculomorphogensis and chemotaxis (Flt1), mitogenesis and differentiation (KDR). Interactions between VEGF-C or (-D) and Flt-4 result in lymphatic proliferation.

Fit1 is a typical receptor tyrosine kinase (RTK), with an extracellular ligand-binding domain, a transmembrane domain and an intracellular kinase domain. Full length of human Flt1 mRNA encodes a 1338 amino acid (aa) residue precursor with a predicted 22 aa residue signal peptide. Mature Flt1 is composed of 737 aa residues of extracelluar domain (ECD), a 22 aa residue transmembrane domain and a 552 aa residue cytoplasmic tyrosine kinas domain. The extracellular domain forms seven Ig-like domains, each having approximately 100 aa residues.

The receptor tyrosine kinase Tie2 (also known as Tek) plays an important role in the development of the embryonic vasculature and persists in adult endothelial cells (ECs) (Schlageger, T. M. etal, Proc. Natl. Acad. Sci. USA, 94;3058-3063, 1997; Dumont, D. et al, Dev. Dyn. 203;80-92, 1995). Tie2 was shown to be upregulated in most of tumors and skin wounds, and in cells under hypoxia conditions, and by its ligands angiopoietin-1 and -2, although they are not directly mitogenic, modulate neovascularization. Tie2 ligands, angiopoietin 3 and 4, were recently confirmed to have functions of promoting blood vessel formation. Angiopoietins and Tie2 are not involved in the initial vasculogenic phase of vascular development as shown for the VEGFs/receptors, but rather participate in vessel sprouting, vessel remodeling, EC migrating (Ang1) and vascular maturation.

Recent reports showed that angiogenesis is an important requirement for the growth and metastasis of tumors (Folkman J., J. Nat. Can. Inst. 82;4-6 1990; Folkman J. Science 235;442-447, 1987; Talks K. L. Brit. J. Haematol. 109;477-489, 2000; Napoleone, F. Kidney Internatl. 56;794-814, 1999). Complete or partial suppression of vascular growth by a number of different strategies has been consistently associated with suppression of tumor expansion and even reduction of tumor burden. However, since angiogenesis is a complex biological process with various factors involved, effective clinical treatment of conditions associated with uncontrolled angiogenesis such as cancer is likely to therapeutically inefficacious if a conventional single-factor approach is employed. Thus, there exists a need for more efficacious therapeutics developed by using non-conventional, innovative approaches using molecules with capacity of binding to several angiogenic factors.

### SUMMARY OF THE INVENTION

The present invention provides novel compositions and methods for treating abnormal cell proliferation and for regulating angiogenesis. In particular, multivalent protein conjugates (MVPs) are constructed to include multiple ligand-binding domains of different receptors and utilized to target multiple, different ligands that are involved in regulation of cell growth and neovascularization. The MVPs of the present invention can be used to treat various conditions associated with abnormal cell proliferation and angiogenesis such as cancer, as well as to promote wound healing.

In one embodiment, the multivalent protein conjugate is represented by the following linear structural formula:

BD₁—L—(BD)ₙ₋₂—L—BDₙ,

wherein BD is a ligand binding domain of a receptor, L is a covalent bond or a linker moiety, and n is an integer from two to about fifty.

In another embodiment, the multivalent protein conjugate is represented by the following structural formula:

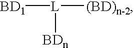

wherein BD is a ligand-binding domain of a receptor, L is a branched linker moiety, and n is an integer from three to about fifty.

BD₁, (BD)ₙ₋₂, and BDₙ may be ligand-binding domains from n different receptors. Alternatively, BD₁, (BD)ₙ₋₂, and BDₙ may be the same ligand binding domain of a receptor. Optionally, where n equals three or more, two or more of BD₁, (BD)ₙ₋₂, and BDₙ may be the same ligand binding domain of a receptor.

Ligand binding domains from a wide variety of receptors may be included. For example, ligand binding domains from cell surface receptors may be linked to form a multivalent protein conjugate of the present invention. Examples of cell surface receptor include, but are not limited to, receptors for growth factors, G-protein coupled receptors, and other cell surface receptor associated with diseases.

Examples of the growth factor include, but are not limited to, epidermal growth factors (EGFs), transferrin, insulin-like growth factor, transforming growth factors (TGFs), and cytokines such as interleukin-1 and interleukin-2. Other cell surface receptor associated with diseases include those that participate in the signal transduction of the formation and development of 1) coronary artery disease such as platelet glycoprotein Iib/IIIa receptor; 2) autoimmune diseases (e.g., mycosis fungoides, generalized postular psoriasis, severe psorisis, and rheumatoid arthritis) such as CD4, CAMPATH-1 and lipid A region of the Gram-negative bacterial lipopolysaccharide; 3) human allergic diseases, such as the receptors of inflammatory mediator protein (e.g., Interleukin-1 (IL-1) and tumor necrosis factor (TNF)), leukotriene, 5-lipoxygenase, and adhesion molecules such as V-CAM/VLA-4.

In a preferred embodiment, BD is a ligand binding domain of a receptor of an angiogenic factor. Examples of the receptor of an angiogenic factor include, but are not limited to those listed in Table I (shown in FIG. 6), such as 1) receptor for angiostatin (angiostatin-R, also called Annexin II), receptor for angiostadin (angiostadin binding protein I), low-affinity receptors for glypicans, receptor for endostatin (endostatin-R), the receptor for endothelin-1 (endothelin-A receptor), receptor for angiocidin (angiocidin-R), the receptor angiogenin (angiogenin-R), receptors for thromospondin-1 and thromospondin-2 (CD36 and CD47), and the receptor for tumstatin (tumstatin-R). The ligand-binding domains of these receptors may be included in the multivalent protein conjugates (MVPs) of the present invention to target multiple anti-angiogenic factors simultaneously, thereby promoting wound healing; 2) receptors for angiogenic growth factors that belong to the family of the receptor tyrosine kinase and are intimately involved in tumor development and metastasis, including receptor for fibrin (VE-cadherin), receptors for VEGF (Flt1 and KDR), receptor for VEGF-C and VEGF-D (Flt4), receptor for VEGF-165 (NP-1 and NP-2), receptors for angiopoeitin-1, -2, -3, and -4 (Tie1 and Tie 2), receptors for FGF (FGF-R1, -R2, -R3 and -R4), receptor for PDGF (PDGF-R), receptor for ephrine A1-5 (Eph A1-8), and receptor for ephrine B1-5 (Eph B1-8). The ligand-binding domains of these receptors may be included in the multivalent protein conjugates (MVPs) of the present invention to target multiple angiogenic growth factors simultaneously for the treatment of various tumors, including benign, malignant and metastatic tumors, and other conditions associated abnormal angiogenesis; 3) G protein coupled receptors such as receptor for sphingosie-1-phosphate or SPP and for lysophosphatidic acid or LSA (edg receptor); 4) cytokine receptors such as receptor for tumor necrosis factor-α or TNF-α (TNF-α receptor) and receptor for interleukin-8 or IL-8 (IL-8 receptor); 5) protease receptors such as receptor for urokinase (urokinase receptor); 6) integrins such as receptor for thromospondin-1 and -2 ((αvβ3 integrin and α2vβ1 integrin ) and receptor for fibronectin (αvβ3 integrin); and 7) matrix metalloprotease.

Optionally, the ligand-binding domain BD may be a ligand-binding domain of Flt1 comprising SEQ ID NO: 26 or 27.

Also optionally, BD is a ligand-binding domain of Tie2 comprising SEQ ID NO: 28, 29 or 30.

Also optionally, when n equals 2 in the multivalent protein conjugate, the amino acid sequence of BD₁ comprises SEQ ID NO: 26 or 27 and the amino acid sequence of BD₂ comprises SEQ ID NO: 28, 29, or 30.

Optionally, the amino acid sequence of the multivalent protein conjugate comprises a sequence selected from the group consisting of 15, 17, 18, and 19.

In addition, BD₁₋ₙ of the multivalent protein conjugate may also be the ligand-binding domain of a nuclear hormone receptor, such as estrogen, androgen, retinoid, vitamin D, glucoccoticoid and progestrone receptors. By linking the ligand-binding domains of various nuclear hormone receptors, the MVP formed is designed to target multiple hormones simultaneously and effectively prevent the binding of these ligands with their cognate receptors in the nucleus, thereby inhibiting pathological effects (e.g., cancer cell growth) resulted from ligand-receptor interactions in the cell.

The ligand-binding domains BD₁₋ₙ may be linked by peptide linkers and expressed as a single fusion protein, or by covalent chemical bonds by chemical synthesis.

The linker moiety L may be a linear peptide linker that connects two BDs covalently and can be incorporated in fusion proteins and expressed in a host cell, such as a prokaryotic cell (e.g., E. coli) and eukaryotic cell (e.g., a mammalian, yeast, or insert cell).

Examples of the linear peptide linker include peptide linkers having at least two amino acid residues such as Gly-Gly [SEQ ID NO: 1], Gly-Ala-Gly [SEQ ID NO: 2], or Gly-Pro-Ala [SEQ ID NO: 3], Gly-Gly-Gly-Gly-Ser [SEQ ID NO: 4] or in andem repeats (preferably 2-4 repeats), etc. The length of the linkers can be from a few to tens of amino acid residues.The peptide linker L is preferably between 2-50 aa in length, more preferably 2-30 aa in length, and most preferably 2-10 aa in length.

Alternatively, the linear peptide linker may be an oligopeptide of from 1 to ˜10 amino acids consisting of amino acids with inert side chains. Suitable oligopeptides include polyglycine, polyserine, polyproline, polyalanine and oligopeptides consisting of alanyl and/or serinyl and/or prolinyl and/or glycyl amino acid residues.

The linker moiety L may also be a branched linker, such as a polypeptide multivalent linker. Preferably, the polypeptide multivalent linker have between about three and about forty amino acid residues, all or some of which provide attachment sites for conjugation with the BDs. Specific examples of such polypeptide multivalent linker include, but are not limited to, polylysines, polyornithines, polycysteines, polyglutamic acid and polyaspartic acid. Optionally, amino acid residues with inert side chains, e.g., glycine, alanine and valine, can be included in the amino acid sequence. The polypeptides can be pennant or cascading.

Optionally, the linker moiety L may be a chemical linker that connects at least two BDs covalently. For example, the chemical linker may be a bifunctional linker, each of which reacts with a BD linearly. Alternatively, the chemical linker may be a branched linker that has a multiplicity of appropriately spaced reactive groups, each of which can react with a functional group of a BD. Suitable reactive groups in a chemical linker include amines, carboxylic acids, alcohols, aldehydes and thiols.

It should be noted that BD₁, BDₙ₋₂, and BDₙ may also associate with each other to form a protein complex via non-covalent interactions such as ionic, hydrogen bonding, Van der Waal's force and hydrophobic interaction. Examples of such protein complexes include, but are not limited to, complexes formed by homo-oligamerization and hetero-oligomerization via some structural units of coiled-coil, leucine-zipper, etc.

Further, a MVP that is a fusion protein of multiple BDs may form a homo- or hetero-oligomer through interaction between an oligomerization unit attached to each MVP. In this way, a MVP complex is formed to acquire a greater diversity of ligand-binding domains. For example, the oligomerization unit is fused to the C-terminus of MVP1 containing BD1 and BD2, while another oligomerization unit is fused to the N-terminus of another MVP2 containing BD3 and BD4. Interactions between the oligomerization units on the two MVPs result in formation of a MVP complex with the two MVPs in a head-to-tail orientation.

Alternatively, the oligomerization unit may be inserted between two BDs in the MVP. Interactions of the oligomerization units on the two MVPs result in formation of a MVP complex with the two MVPs potentially interacting with each other in parallel, or in a cruciform conformation.

The oligomerization unit may be a naturally occurring or synthetic polypeptide. Preferably, the oligomerization unit is non-immunogenic to a human body. For example, the oligomerization unit may be derived from the dimerization unit of receptors for opioid, muscarinic, dopamine, serotonin, adenosine/dopamine, and GABA-B.

The oligomerization unit included in each MVP may be the same or different. For example the oligomerization unit on MVP1 may be a leucine zipper domain from the nuclear oncoprotein Jun while the oligomerization unit on MVP1 may be a leucine zipper domain from the nuclear oncoprotein Fos. Alternatively, a heterodimer MVP complex may be formed between MVP1 and MVP2, including the leucine zipper domain of the proto-oncoproteins Myc and Max, respectively.

In yet another embodiment, the multivalent protein conjugate may further comprise a tag sequence (Tag), resulting in a structure having the following general formula:

Tag—BD₁—L—(BD)ₙ₋₂—L—BDₙ

or

BD₁—L—(BD)ₙ₋₂—L—BDₙ—Tag

In one embodiment, Tag may be a protein or peptide that serves as a recognition site for the immune system. For example, Tag may be a fragment of a human immunoglobulin, e.g., the constant region (Fe) of human IgG1. Tag may also be an affinity tag for the convenience of detection and purification of the conjugate. Examples of the affinity tag include, but are not limited to, a polyhistidine tract, polyarginine or polylysine, glutathione-S-transferase (GST), maltose binding protein (MBP), a portion of staphylococcal protein A (SPA), FLAG, virus hemoagglutin (HA) and various immunoaffinity tags (e.g. protein A) and epitope tags such as those recognized by the EE (Glu-Glu) antipeptide antibodies.

Optionally, the multivalent protein conjugate may include tag sequences in both the N-terminus (Tag_{N}) and the C-terminus (Tag_{C}) of the conjugate, resulting in a structure having the following general formula:

Tag_{N}—BD₁—L—(BD)ₙ₋₂—L—BDₙ—Tag_{C}

Alternatively, Tag may be positioned between the ligand-binding domains (e.g., between BD₁ and BD₂), resulting in the structure with the following general formula:

BD₁—L—Tag—(BD)ₙ₋₂—L—BDₙ,

BD₁—L—(BD)ₙ₋₂—Tag—L—BDₙ,

BD₁—L—Tag—L—(BD)ₙ₋₂−L−BDₙ,

or

Tag—BD₁—L—Tag—L—(BD)ₙ₋₂—L—BDₙ,

wherein BD is a ligand-binding domain of a receptor, L is a covalent bond or a linker moiety, Tag is a tag peptide sequence, and n is an integer from two to about fifty.

Tag in this structure can serve as a linker linking two ligand-binding domains.

Examples of Tag includes, but are not limited, the constant region (Fc) of human IgG1, IgG2 or IgG4, a polyhistidine tract, polyarginine, polylysine, glutathione-S-transferase (GST), maltose binding protein, a portion of staphylococcal protein A, FLAG, a myc tag, virus hemaagglutin and various immunoaffinity tags, and an EE tag. Particularly, Tag is human IgG1 Fc having an amino acid sequence of SEQ ID NO: 31.

According to the present invention, the MVP can not only be used as a monotherapy to treat various diseased conditions, but also in conjunction with other therapeutic agents for the treatment.

In one embodiment, the MVP is used in combination with an anti-angiogenesis agent for the treatment of diseases associated with abnormal angiogenesis.

Examples of anti-angiogenesis agents include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,1-3,4-dehydroproline, thiaproline], α, α-dipyridyl, β-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta.-1-anticollagenase-serum, α2-antiplasmin, bisantrene, lobenzarit disodium, n-(2-carboxyphenyl-4-chloroanthronilic acid disodium or “CCA”, thalidomide; angostatic steroid, cargboxynaminolmidazole; metalloproteinase inhibitors such as BB94. Other anti-angiogenesis agents include antibodies, such as monoclonal antibodies against these angiogenic growth factors: bFGF, aFGF, FGF-5, VEGF isoforms, VEGF-C, HGF/SF and Ang-1/Ang-2.

The compositions of the present invention may be used to treat a wide variety of indications for which the multivalent protein conjugate has therapeutic activity. Such indications include, but are not limited to, restenosis (e.g. coronary, carotid, and cerebral lesions), benign tumors, a various types of cancers such as primary tumors and tumor metastasis, abnormal stimulation of endothelial cells (atherosclerosis), insults to body tissue due to surgery, abnormal wound healing, abnormal angiogenesis, diseases that produce fibrosis of tissue, muscular degeneration, repetitive motion disorders, disorders of tissues that are not highly vascularized, and proliferative responses associated with organ transplants.

Examples of benign tumors include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

Specific types of cancers include, but are not limited to, leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Diseases associated with abnormal angiogenesis include, but are not limited to, rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Examples of retinal/choroidal neovascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disese, systemic lupus erythematosis, retinopathy of prematurity, Eales disease, diabetic retinopathy, macular degeneration,, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the ankle) and diseases caused by the abnormal proliferation of fibrovascular or fibrous tissue including all forms of proliferative vitreoretinopathy.

Examples of corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

### BRIEF DESCRIPTION OF THE FIGURES

FIG. 1 illustrates two embodiments of a linear MVP.

FIG. 2 illustrates an embodiment of a MVP wherein the ligand-binding domains of receptors are linked by a branched linker.

FIG. 3 illustrates an embodiment of a MVP wherein the ligand-binding domains of receptors are linked by a cascading polypeptide multivalent linker.

FIG. 4 illustrates an embodiment of a MVP wherein the ligand-binding domains of receptors are linked by a pennant polypeptide multivalent linker.

FIG. 5A illustrates an embodiment of a MVP complex wherein two MVPs interact with each in a head-to-tail orientation through an oligomerization unit attached to the end of each conjugate.

FIG. 5B illustrates an embodiment of a MVP complex wherein two MVPs interact with each in a parallel orientation through an oligomerization unit inserted between two ligand-binding domains of receptors on each conjugate.

FIG. 5C illustrates an embodiment of a MVP complex wherein two MVPs interact with each in a cruciform conformation through an oligomerization unit inserted between two ligand-binding domains of receptors on each conjugate.

FIG. 6 is Table I listing examples of receptors and their ligands that are involved in regulation of angiogenesis.

FIG. 7A shows the design of MVP-A (also termed “2FT/A”) containing ligand-binding domains of Flt1 and Tie2 (Flt1-D₂ -Tie2-D₁₋₃-Fc) and lists the DNA sequence [SEQ ID NO: 14] and amino acid sequence [SEQ ID NO: 15] of MVP-A.

FIG. 7B shows the design of MVP-B containing ligand-binding domains of Flt1 and Tie2 (Flt1-D₂₋₃-GG-Tie2-D₁₋₃-Fc) and lists the DNA sequence [SEQ ID NO: 16] and amino acid sequence [SEQ ID NO: 17] of MVP-B.

FIG. 7C shows the design of MVP-C containing ligand-binding domains of Flt1 and Tie2 (Flt1-D₂₋₃-Tie2-D₁₋₃-Fc) and lists the amino acid sequence [SEQ ID NO: 18] of MVP-C.

FIG. 7D shows the design of MVP-D containing ligand-binding domains of Tie2 and Flt1 (Tie2-D₁₋₃-Fc-Flt1-D₂₋₃) and lists the amino acid sequence [SEQ ID NO: 19] of MVP-D.

FIG. 7E lists amino acid sequences of ligand-binding domains of Flt1 and Tie2, and sequences of HuIgG1 Fc and secretory leader sequences of Tie2.

FIG. 8 is a diagram showing a plasmid for expressing the multivalent protein conjugate 2FT/A. The functional domain of each component is labeled in the diagram.

FIG. 9 shows an agarose gel image showing the restriction map of the plasmid expressing 2FT/A with the Dhfr and Kozak sequences.

FIG. 10 shows a SDS-PAGE gel showing the purified 2FT/A visualized by the silver staining (right panel) and Western blot (left panel).

FIG. 11 shows results from a cell proliferation assay, indicating that 2FT/A could block VEGF-induced growth of BBE cells.

FIG. 12 shows results from a cell proliferation assay, indicating that 2FT/A could block bFGF-induced VEGF release which caused cell growth reduction via an endocrine loop.

### DETAILED DESCRIPTION OF THE INVENTION

The present invention discloses a non-conventional methodology that can be utilized to treat diseased conditions resulted from interactions between multiple receptors and their cognate ligands, in particular, from the interactions between angiogenic receptors and ligands. The methodology of the present invention capitalizes on the intrinsic properties of a receptor having a ligand-binding domain that is substantially structurally and functionally separable from other domains of the receptor. Employing this approach, a multivalent protein conjugate is constructed, in which at least two ligand-binding domains of two different receptors are preferably linked covalently. Alternatively, the multivalent protein conjugate may also contain multiple copies of the same ligand-binding domain.

Not wishing to be bound by the theory, it is believed that the multivalent protein conjugate should exert a higher therapeutic efficacy by regulating the activity of multiple receptors simultaneously. It is also believed that by targeting multiple, different receptors that participate in the same or different stage of disease formation and development, resistance to a drug targeting a single receptor may be circumvented.

In one embodiment, the multivalent protein conjugate is represented by the following linear structural formula:

BD₁—L—(BD)ₙ₋₂—L—BDₙ,

wherein BD is a ligand binding domain of a receptor, L is a covalent bond or a linker moiety, and n is an integer from two to about fifty.

Alternatively, BD₁, (BD)ₙ₋₂, and BDₙ may associate with each other to form a protein complex via non-covalent interactions such as ionic, hydrogen bonding, Van der Waal's force and hydrophobic interaction. Examples of such protein complexes include, but are not limited to, complexes formed by homo-oligamerization and hetero-oligomerization via structural units of coiled-coil, leucine-zipper, etc.

In a preferred embodiment, BD₁, (BD)ₙ₋₂, and BDₙ are ligand binding domains from n different receptors. Alternatively, BD₁, (BD)ₙ₋₂, and BDₙ may be the same ligand binding domain of a receptor. Optionally, where n equals three or more, two or more of BD₁, (BD)ₙ₋₂, and BDₙ may be the same ligand binding domain of a receptor.

In another embodiment, the multivalent protein conjugate is represented by the following structural formula:

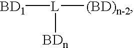

wherein BD is a ligand binding domain of a receptor, L is a branched linker moiety, and n is an integer from three to about fifty.

In a preferred embodiment, BD₁, (BD)ₙ₋₂, and BDₙ are ligand binding domains from n different receptors. Optionally, two or more of BD₁, (BD)ₙ₋₂, and BDₙ may be the same ligand binding domain of a receptor.

According to the present invention, a multivalent protein conjugate is constructed that include at least two ligand-binding domains of receptors. The ligand binding domains may be linked by peptide linkers and expressed as a single fusion protein, or by covalent chemical bonds by chemical synthesis. The multivalent protein conjugate may further comprise a tag sequence (Tag), resulting in a structure having the following general formula:

Tag—BD₁—L—(BD)ₙ₋₂—L—BDₙ

or

BD₁—L—(BD)ₙ₋₂—L—BDₙ—Tag

In one embodiment, Tag may be a protein or peptide that serves as a recognition site for the immune system. For example, Tag may be a fragment of a human immunoglobulin, e.g., the constant region (Fc) of human IgG1, IgG2 or IgG4. The Fc fragment can be recognized by Fe receptor positive monocytes and be cleared by the monocytes mediated process. Tag may also be an affinity tag for the convenience of detection and purification of the conjugate. Examples of the affinity tag include, but are not limited to, a polyhistidine tract, polyarginine or polylysine, glutathione-S-transferase (GST), maltose binding protein (MBP), a portion of staphylococcal protein A (SPA), FLAG, virus hemoagglutin (HA), myc tag and various immunoaffinity tags (e.g. protein A) and epitope tags such as those recognized by the EE (Glu-Glu) antipeptide antibodies.

Optionally, the multivalent protein conjugate may include tag sequences in both the N-terminus (Tag_{N}) and the C-terminus (Tag_{C}) of the conjugate, resulting in a structure having the following general formula:

Tag_{N}—BD₁—L—(BD)ₙ₋₂—L—BDₙ—Tag_{C}

In a preferred embodiment, human IgG Fc fragment is used as the Tag and the multiple valent protein (MVP) is expressed as fusion protein. After purification, the Fc tag is either removed by pre-designed protease cleavage site such enterokinase, thrombin, urokinase, etc. or remains attached. The function of MVP can be assayed in vitro for binding to corresponding ligands and effects on angiogenesis.

By combining the ligand binding domains of multiple receptors into a single chemical entity, the multivalent protein conjugate generated is believed to possess several advantages over a protein containing only a single binding domain of a receptor. First, since the multivalent protein conjugate contains the ligand-binding domains of multiple receptors, the conjugate can target multiple cognate ligands of these receptors simultaneously. Compared with a “monotherapy” involving a therapeutic protein containing only a single ligand-binding domain, the multivalent conjugate should have a much higher therapeutic index. Further, this “cocktail” approach may prevent or circumvent resistance developed by the tumors in response to the monotherapy, thereby enhancing the therapeutic efficacy of the conjugate.

In addition, avidity of the multivalent protein conjugate may be increased by linking multiple ligand binding domains. It is believed that this process may mimic the natural assembly of multiple immunoglobulin IgMs during the primary immune response. The low affinity of IgM is compensated by its pentameric structure, resulting in a high avidity toward repetitive antigenic determinants present on the surface of bacteria or viruses. Thus, the binding affinity of the ligand with its cognate receptor's binding domain may be enhanced by multivalent binding of multiple ligands to the conjugate, which in turn further enhances therapeutic efficacy of the conjugate.

1. The Ligand Binding Domain (BD) of Receptors

Ligand binding domains from a wide variety of receptors may be included. For example, ligand binding domains from cell surface receptors may be linked to form a multivalent protein conjugate of the present invention. Examples of cell surface receptor include, but are not limited to, receptors for growth factors and other cell surface receptor associated with diseases.

Examples of the growth factor include, but are not limited to, epidermal growth factors (EGFs), transferrin, insulin-like growth factor, transforming growth factors (TGFs), interleukin-1, and interleukin-2. For example, high level expression of EGF receptors have been found in a wide variety of human epithelial primary tumors. TGF-α have been found to mediate an autocrine stimulation pathway in cancer cells.

Other cell surface receptor associated with diseases include those that participate in the signal transduction of the formation and development of 1) coronary artery disease such as platelet glycoprotein Iib/IIIa receptor; 2) autoimmune diseases (e.g., mycosis fungoides, generalized postular psoriasis, severe psorisis, and rheumatoid arthritis) such as CD4, CAMPATH-1 and lipid A region of the Gram-negative bacterial lipopolysaccharide; 3) human allergic diseases, such as the receptors of inflammatory mediator protein (e.g., Interleukin-1 (IL-1) and tumor necrosis factor (TNF)), leukotriene, 5-lipoxygenase, and adhesion molecules such as V-CAM/VLA-4.

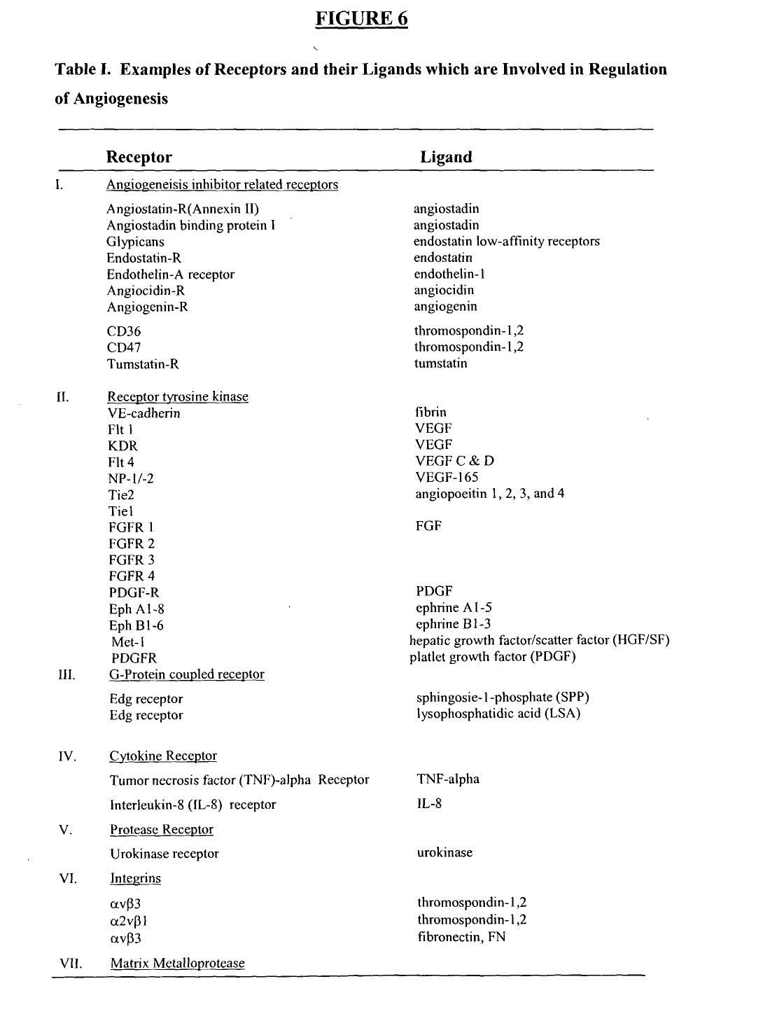

In a preferred embodiment, BD is a ligand binding domain of a receptor of an angiogenic factor. Examples of the receptor of an angiogenic factor include, but are not limited to those listed in Table I as shown in FIG. 6.

As listed in Table I, many receptors have been identified for binding to their cognate ligands. In particular, receptors for protein factors that have anti-angiogenic effects include, but are not limited to, receptor for angiostatin (angiostatin-R, also called Annexin II), receptor for angiostadin (angiostadin binding protein I), low-affinity receptors for glypicans, receptor for endostatin (endostatin-R), the receptor for endothelin-1 (endothelin-A receptor), receptor for angiocidin (angiocidin-R), the receptor angiogenin (angiogenin-R), receptors for thromospondin-1 and thromospondin-2 (CD36 and CD47), and the receptor for tumstatin (tumstatin-R). The ligand-binding domains of these receptors may be included in the multivalent protein conjugate (MVP) of the present invention to target multiple anti-angiogenic factors simultaneously. Through binding to these anti-angiogenic factors, the MVP can efficiently inhibit anti-angiogenic effects of these factors and promote angiogenesis. Such an effect is particular desirable in wound healing.

Also listed in Table I are receptors for angiogenic growth factors that belong to the family of the receptor tyrosine kinase and are intimately involved in tumor development and metastasis, including receptor for fibrin (VE-cadherin), receptors for VEGF (Flt1 and KDR), receptor for VEGF-C and VEGF-D (Flt4), receptor for VEGF-165 (NP-1 and NP-2), receptors for angiopoietin-1, -2, -3, and -4 (Tie1 and Tie 2), receptors for FGF (FGF-R1, -R2, -R3 and -R4), receptor for PDGF (PDGF-R), receptor for ephrine A1-5 (Eph A1-8), and receptor for ephrine B1-5 (Eph B1-8). The ligand-binding domains of these receptors may be included in the multivalent protein conjugate (MVP) of the present invention to target multiple angiogenic growth factors simultaneously. Through binding to these angiogenic growth factors, the MVP can efficiently inhibit angiogenic effects of these growth factors and suppress angiogenesis. Such an effect is particular desirable in the treatment of various tumors, including benign, malignant and metastatic tumors, and other conditions associated abnormal angiogenesis.

Also listed in Table I are G protein coupled receptors such as receptor for sphingosie-1-phosphate or SPP and for lysophosphatidic acid or LSA (edg receptor), cytokine receptors such as receptor for tumor necrosis factor-α or TNF-α (TNF-α receptor) and receptor for interleukin-8 or IL-8 (IL-8 receptor), protease receptors such as receptor for urokinase (urokinase receptor), and integrins such as receptor for thromospondin-1 and -2 (αvβ3 integrin and α2vβ1 integrin) and receptor for fibronectin (αvβ3 integrin), and matrix metalloprotease. The ligand-binding domains of these receptors and proteases may be included in the multivalent protein conjugate (MVP) of the present invention to target their cognate ligands, thereby reducing the pathological effects resulted from interactions between these proteins and their ligands.

The BD of the multivalent protein conjugate may also be the ligand binding domain of a nuclear hormone receptor, such as estrogen, androgen, retinoid, vitamin D, glucoccoticoid and progestrone receptors.

Nuclear hormone receptor proteins form a class of ligand activated proteins that, when bound to specific sequences of DNA serve as on-off switches for transcription within the cell nucleus. These switches control the development and differentiation of skin, bone and behavioral centers in the brain, as well as the continual regulation of reproductive tissues. Interactions between nuclear hormone receptors and their cognate ligands have been implicated in the initiation and development of various forms of cancer such as breast, prostate, bone, and ovarian cancer.

At the molecular level, nuclear hormone receptors are ligand-activated transcription factors that regulate gene expression by interacting with specific DNA sequences upstream of their target genes. A two-step mechanism of action was proposed for these receptors based upon the observation of an inactive and an active state of the receptors. The first step involves activation through binding of the hormone; and the second step consists of receptor binding to DNA and regulation of transcription. A hormone response element (HRE) is a specific DNA sequence that a receptor recognizes with markedly increased affinity and typically contains two consensus hexameric half-sites. The identity of a response element resides in three features: the sequence of the base pairs in the half-site, the number of base pairs between the half-sites and the relative orientation of the two half-sites. Thus each receptor protein dimer that binds the DNA has to recognize the sequence, spacing and orientation of the half-sites within their response element.

The nuclear hormone receptor proteins are composed of several domains which are differentially conserved between the various receptors and have different roles: a variable N-terminal region, a conserved DNA binding domain (DBD), a variable hinge region, a conserved ligand binding domain (LBD), and a variable C-terminal region.

The central DBD is responsible for targeting the receptors to their hormone response elements (HRE). The DNA binding domain, classified as a type-II zinc finger motif, has two subdomains, each containing a zinc ion coordinated by four cysteine residues, followed by an alpha-helix. The DBD binds as a dimer with each monomer recognizing a six base pair sequence of DNA. The reading helix of each monomer makes sequence specific contacts in the major groove of the DNA at each half-site. These contacts allow the dimer to read the sequence, spacing and orientation of the half-sites within its response element, and thus discriminate between sequences. These proteins exhibit, however, a flexibility in recognizing DNA sequences and also accept a variety of amino-acid substitutions in their reading helix without abolishing binding.

The LBD participates in several activities including hormone binding, homo- and/or heterodimerization, formation of the heat-shock protein complex and transcriptional activation and repression. The binding of the hormone induces conformational changes that seem to control these properties and influence gene expression. The conformational changes that accompany the transition between the liganded and unliganded forms of the nuclear hormone receptors affect dramatically their affinity for other proteins.

According to the present invention, since the ligand binding domain (LBD) of a nuclear hormone receptor is structurally separable from the other domains of the receptor, LBDs of multiple nuclear hormone receptors may be linked to form a multivalent protein conjugate. The conjugate may be used to treat or prevent various forms of cancers or other disease conditions associated with interactions between the nuclear hormone receptors and their cognate ligands.

2. The Linker (L) Between the BDs

The linker moiety L in the multivalent protein conjugate is used to covalently connect two or more individual domains of the multivalent proteins. The linker is preferred to be one that increases flexibility of the linked binding domains (BDs) and not to interfere significantly with the structure of each functional BD within the whole conjugate. More preferably, immunogenicity of each functional BD within the conjugate does not deviate from that of the native form BD situated in its cognate protein.

1) Peptide Linker

The linker moiety L may be a linear peptide linker that connects two BDs covalently and can be incorporated in fusion proteins and expressed in a host cell, such as a prokaryotic cell (e.g., E. coli) and eukaryotic cell (e.g., a mammalian, yeast, or insert cell).

Examples of the linker include peptide linkers having at least two amino acid residues such as Gly-Gly [SEQ ID NO: 1], Gly-Ala-Gly [SEQ ID NO: 2], or Gly-Pro-Ala [SEQ ID NO: 3], etc. The length of the linkers can be from a few to tens of amino acid residues. The peptide linker L is preferably between 2-50 aa in length, more preferably 2-30 aa in length, and most preferably 2-10 aa in length.

In one embodiment, the linear peptide linker is an oligopeptide of from 1 to ˜10 amino acids consisting of amino acids with inert side chains. Suitable oligopeptides include polyglycine, polyserine, polyproline, polyalanine and oligopeptides consisting of alanyl and/or serinyl and/or prolinyl and/or glycyl amino acid residues.

In one particular embodiment, the linker may be the G₄S peptide linker: Gly-Gly-Gly-Gly-Ser [SEQ ID NO: 4], or the G₄S linker in tandem repeats, preferably 2-4 repeats.

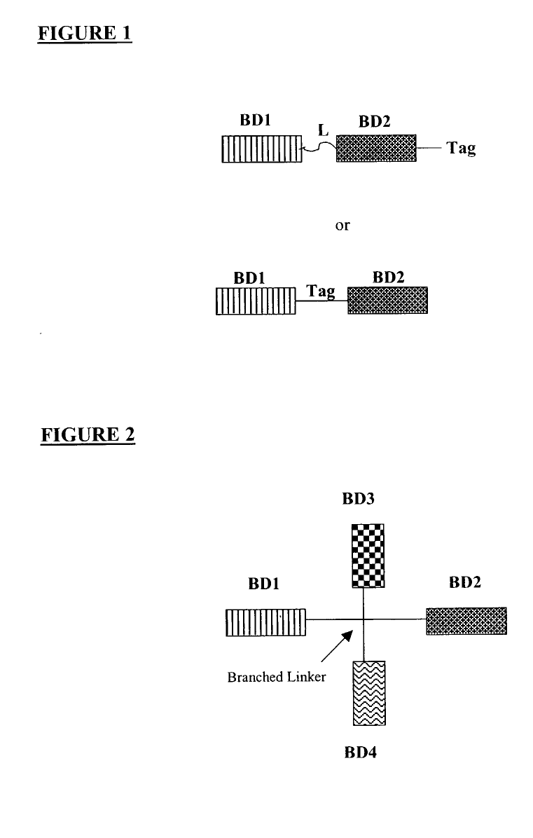

FIG. 1 shows examples of the multivalent protein conjugate in which the BDs are linked by linear peptide linkers. As illustrated in FIG. 1, the ligand-binding domains from two different receptors, BD1 and BD2, are linked through their C-terminus and N-terminus, respectively, in tandem by a linear peptide linker L. The recombinant MVP formed can be produced in large amounts by expressing it as a fusion protein in cell culture.

Alternatively, the linker moiety L may be a polypeptide multivalent linker. As illustrated in FIG. 2, this type of linker has branched “arms” that link with multiple BDs in a non-linear fashion. Examples of suitable polypeptide multivalent backbones include, but are not limited to, those linkers disclosed in Tam (1996) Journal of Immunological Methods 196:17, the entire teachings of which are incorporated herein by reference. As illustrated in FIG. 2, the ligand-binding domains from four different receptors, BD1, BD2, BD3 and BD4, are linked together by the four “arms” of a branched linker to form a MVP of the present invention.

The branched linker may be a polypeptide multivalent linker. Preferably, the polypeptide multivalent linker have between about three and about forty amino acid residues, all or some of which provide attachment sites for conjugation with the BDs. More preferably, the linker has between about two and about twenty attachment sites, which are often functional groups located in the amino acid residue side chains. However, alpha amino groups and alpha carboxylic acids can also serve as attachment sites.

Specific examples of such polypeptide multivalent linker include, but are not limited to, polylysines, polyornithines, polycysteines, polyglutamic acid and polyaspartic acid. Optionally, amino acid residues with inert side chains, e.g., glycine, alanine and valine, can be included in the amino acid sequence. The polypeptides can be pennant or cascading.

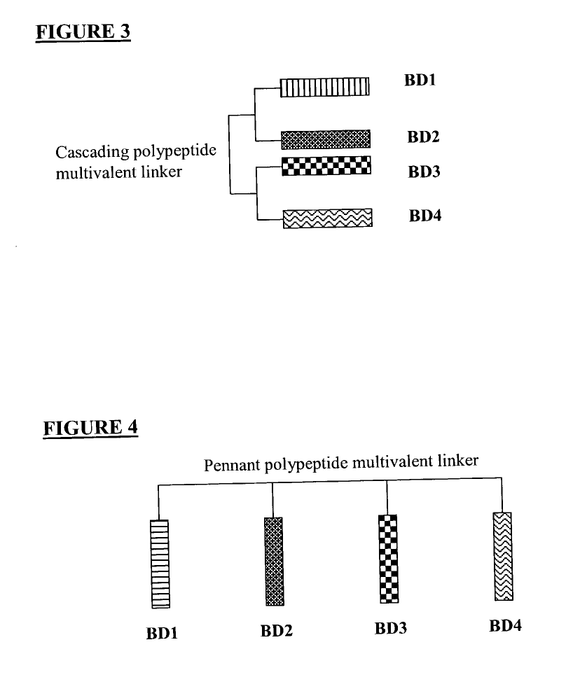

FIG. 3 illustrates an example of a “cascading” polypeptide multivalent linker which is branched with at least some of the amide bonds formed between the side chain functional group of one amino acid residue and the alpha amino group or alpha carboxylic acid group of the next amino acid residue. For example, at least some of the amide bonds of a cascading polylysine are formed between the epsilon amine group of a lysine residue and the carboxylic acid residue of the next lysine residue. As illustrated in FIG. 3, this type of linker can be used to link the ligand-binding domains from four different receptors, BD1, BD2, BD3 and BD4, to form a MVP of the present invention.

FIG. 4 illustrates an example of a “pennant” polypeptide multivalent linker. As with polypeptides typically found in nature, the amide bonds of a pennant polypeptide are formed between the alpha amine of one amino acid residue and the alpha carboxylic acid of the next amino acid residue. When n is less than five, there are typically 0-6 amino acids between attachment sites; when n is greater than five, there are typically 1-6 amino acids between attachment sites. As illustrated in FIG. 4, this type of linker can be used to link the ligand-binding domains from four different receptors, BD1, BD2, BD3 and BD4, to form a MVP of the present invention.

2) Chemical Linker

The linker moiety L may be a chemical linker that connects at least two BDs covalently. Preferably, the chemical linker is biocompatible and, after attachment of the BDs, are suitable for parenteral or oral administration.

For a multivalent protein conjugate that contains BDs linked linearly, the chemical linker may be a bifunctional linker, each of which reacts with a BD. Alternatively, the chemical linker may be a branched linker that has a multiplicity of appropriately spaced reactive groups, each of which can react with a functional group of a BD. The branched linker typically has molecular weights less than about 20,000 atomic mass units and typically comprises between two to about a hundred attachment sites. Not all attachment sites need be occupied.

Reactive functional groups in a branched linker serve as attachment sites for the BDs. Attachment sites are “appropriately spaced” when steric hindrance does not substantially interfere with forming covalent bonds between some of the reactive functional groups and the peptide.

Suitable reactive groups in a chemical linker include amines, carboxylic acids, alcohols, aldehydes and thiols. An amine group in a chemical linker can form a covalent bond with the C-terminal of a BD or a carboxylic acid functional group on the side chain of an amino acid residue of a BD. A carboxylic acid group or an aldehyde in a chemical linker forms a covalent bond with the N-terminus of a BD or an amine group on the side chain of an amino acid residue of a BD. An alcohol group in a chemical linker can form a covalent bond with the C-terminus of a BD or a carboxylic acid group on the side chain of an amino acid residue of a BD. A thiol group in a chemical linker can form a disulfide bond with a cysteine on the side chain of an amino acid residue of a BD. Covalent Bonds can also be formed between other reactive functional groups in the chemical linker and appropriate functional groups in the amino acid side chains of the attached BDs. The functionality which connects each BD to the chemical linker can be different, but is preferably the same for all BDs.

For example, the linker may be

M₁—(CH₂)ₘ—M₂

or

M₁—PEG—M₂

Wherein M₁ and M₂ are each a functional group which is connected by a covalent bond to a suitable functional group residue in a BD, CH₂ is a methylene group, m is an integer from two to about 20, and PEG is polyethylene glycol.

Examples of M₁ and M₂ include: 1) the residue of an alcohol group which forms an ester with the residue of a carboxylic acid group in a BD; 2) the residue of an amine group which forms an amide with the residue of a carboxylic acid group in a BD; 3) the residue of a carboxylic acid or aldehyde group which forms an amide with the residue of an amine in a BD; or 4) the residue of a thiol group which forms a disulfide bond with the residue of a thiol group in a BD.

3) MVP Complex Formed via Oligomerization

Also according to the present invention, the ligand-binding domains (BDs) of the same or different receptors may form a multivalent protein conjugate (MVP) complex via non-covalent interactions between an oligomerization unit fused with the BD. The fusion protein formed by a BD and the oligomerization unit may be expressed by a single vector in the cell where a multivalent homo-oligomer of the same BD is formed. Alternatively, several expression vectors each of which encodes a fusion protein formed by a different BD and the same oligomerization unit may be co-transfected into the cell where a multivalent hetero-oligomer of the different BDs is formed. Further, a MVP that is a fusion protein of multiple BDs as described in detail above may form a homo- or hetero-oligomer through interaction between the oligomerization unit attached to each MVP. In this way, an even more complex MVP is formed, which should enhance the avidity and diversity of the MVP.

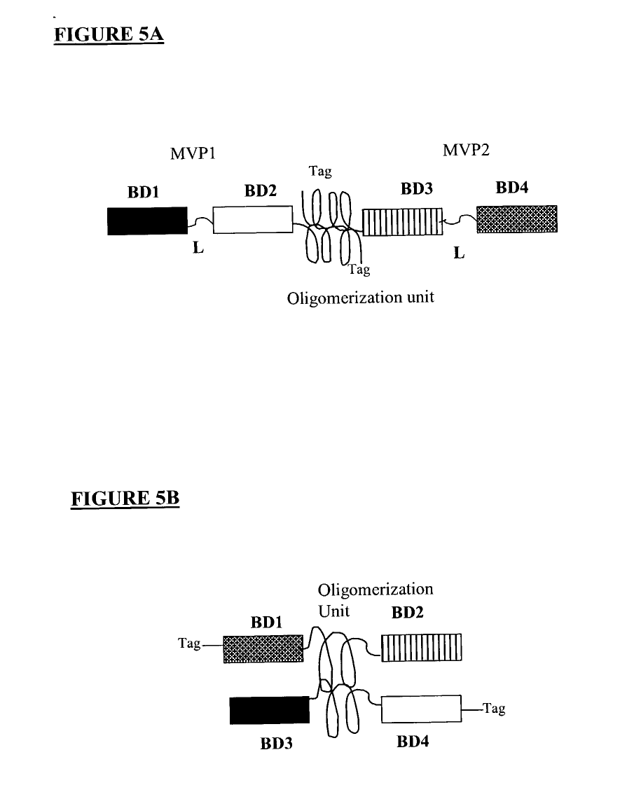

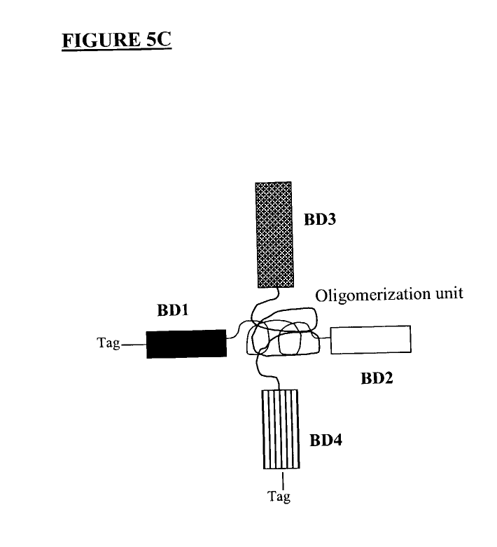

FIGS. 5A-C illustrate various ways in which MVPs having at least 2 different BDs can form an MVP complex through an oligomerization unit included in the MVP. As illustrated in FIG. 5A, an oligomerization unit is fused to the C-terminus of the MVP containing BD1 and BD2, while another oligomerization unit is fused to the N-terminus of the MVP containing BD3 and BD4. When MVP1 and MVP2 are expressed in the cells, through oligomerization of the oligomerization units on the two MVPs, a MVP complex is formed with the two MVPs in a head-to-tail interaction.

Alternatively, the oligomerization unit may be inserted between two BDs in the MVP. As illustrated in FIG. 5B, an oligomerization unit is inserted between BD1 and BD2 of MVP1 and also serves as the linker L between these two BDs. Likewise, another oligomerization unit is inserted between BD3 and BD4 of MVP2 and also serves as the linker L between these two BDs. When MVP1 and MVP2 are expressed in the cells, through oligomerization of the oligomerization units on the two MVPs, a MVP complex is formed with the two MVPs potentially interacting with each other in parallel.

It is also plausible that MVP1 and MVP2 may interact with each other in a cruciform conformation through the oligomerization units inserted between BD1 and BD2, and BD1 and BD2, respectively. As illustrated in FIG. 5C, a MVP complex adopting a cruiform conformation is formed between MVP1 and MVP2 via interactions between the oligomerization units between the two BDs on each MVP.

The oligomerization unit may be a naturally occurring or synthetic polypeptide. Preferably, the oligomerization unit is non-immunogenic to a human body. For example, the oligomerization unit may be derived from the dimerization unit of receptors for opioid, muscarinic, dopamine, serotonin, adenosine/dopamine, and GABA-B.

The oligomerization unit included in each MVP may be the same or different. For example the oligomerization unit on MVP1 may be a leucine zipper domain from the nuclear oncoprotein Jun while the oligomerization unit on MVP1 may be a leucine zipper domain from the nuclear oncoprotein Fos. Kouzarides and Tiff (1989) “Behind the Fos and Jun leucine zipper” Cancer Cells 1: 71-76. Heterodimerization between Jun and Fos should allow the formation of the complex between MVP1 and MVP2.

Alternatively, a heterodimer MVP complex may be formed between MVP1 and MVP2, including the leucine zipper domain of the proto-oncoproteins Myc and Max, respectively. Luscher and Larsson (1999) “The basic region/helix-loop-helix/leucine zipper domain of Myc proto-oncoproteins: function and regulation” Ongogene 18:2955-2966.

3. Combination Therapy of MVP

The multivalent protein conjugate (MVP) of the present invention may also be used in combination with other therapeutic agents to treat cancer and other diseases associated abnormal cell proliferation and angiogenesis.

A wide variety of therapeutic agents may have a therapeutic additive or synergistic effect with the multivalent protein conjugate. Such therapeutic agents may be hyperplastic inhibitory agents that addictively or synergistically combine with the multivalent protein conjugate to inhibit undesirable cell growth, such as inappropriate cell growth resulting in undesirable benign conditions or tumor growth. Examples of such therapeutic agents include, but are not limited to, alkylating agents, antibiotic agents, antimetabolic agents, hormonal agents, plant-derived agents, and biologic agents.

The alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. Combination therapy including the multivalent protein conjugate and the alkylating agent may have therapeutic synergistic effects on cancer and reduce sides affects associated with these chemotherapeutic agents.

The antibiotic agents are a group of drugs that produced in a manner similar to antibiotics as a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interferes with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. Combination therapy including the multivalent protein conjugate and the antibiotic agent may have therapeutic synergistic effects on cancer and reduce sides affects associated with these chemotherapeutic agents.

The antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including the multivalent protein conjugate and the antimetabolic agent may have therapeutic synergistic effects on cancer and reduce sides affects associated with these chemotherapeutic agents.

The hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. Combination therapy including the multivalent protein conjugate and the hormonal agent may have therapeutic synergistic effects on cancer and reduce sides affects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), camptothecins including 20(S)-camptothecin, 9-nitro-20(S)camptothecin and 9-amino-20(S) camptothecin, taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including the multivalent protein conjugate and the plant-derived agent may have therapeutic synergistic effects on cancer and reduce sides affects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including the multivalent protein conjugate and the biologic agent may have therapeutic synergistic effects on cancer, enhance the patient's immune responses to tumorigenic signals, and reduce potential sides affects associated with this chemotherapeutic agent.

Cytokines possess profound immunomodulatory activity. Some cytokines such as interleukin-2 (IL-2, aldesleukin) and interferon α (IFN-α) demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with the multivalent protein conjugate include, but are not limited to, interleukin 2 (IL-2), and interleukin 4 (IL-4), interleukin 12 (IL-12).

Interferon a include more than 23 related subtypes with overlapping activities, all of the IFN-□ subtypes within the scope of the present invention. IFN-α has demonstrated activity against many solid and hematologic malignancies, the later appearing to be particularly sensitive. Examples of interferons that may be used in conjunction with the multivalent protein conjugate include, but are not limited to, interferon α, interferon β (fibroblast interferon) and interferon γ (fibroblast interferon).

Other cytokines that may be used in conjunction with the multivalent protein conjugate include those cytokines that exert profound effects on hematopoiesis and immune functions. Examples of such cytokines include, but are not limited to erythropoietin (epoietin α), granulocyte-CSF (filgrastin), and granulocyte, macrophage-CSF (sargramostim). These cytokines may be used in conjunction with the multivalent protein conjugate to reduce chemotherapy-induced myelopoietic toxicity.

Other immuno-modulating agents other than cytokines may also be used in conjunction with the multivalent protein conjugate to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including the multivalent protein conjugate and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and maligant CD20⁺ pre-B and mature B cells. RITUXAN® is used as single agent for the treatment of patients with relapsed or refractory low-grade or follicular, CD20+, B cell non-Hodgkin's lymphoma. Combination therapy including the multivalent protein conjugate and RITUXAN® may have therapeutic synergistic effects not only on lymphoma, but also on other forms or types of malignant tumors.

Tumor suppressor genes are genes that function to inhibit the cell growth and division cycles, thus preventing the development of neoplasia. Mutions in tumor suppressor genes cause the cell to ignore one or more of the components of the network of inhibitory signals, overcoming the cell cycle check points and resulting in a higher rate of controlled cell growth—cancer. Examples of the tumor suppressor genes include, but are not limited to, DPC-4, NF-1, NF-2, RB, p53, WT1, BRCA1 and BRCA2.

DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions. Combination therapy including the multivalent protein conjugate and tumor suppressor may have therapeutic synergistic effects on patients suffering from various forms of cancers.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to teh tumor cell, TAAs provide targets for the immune system to recognize and cause their destruction. Example of TAAs include, but are not limited to gangliosides (GM2), prostate specific antigen (PSA), α-fetoprotein (AFP), carcinoembryonic antigen (CEA) (produced by colon cancers and other adenocarcinomas, e.g. breast, lung, gastric, and pancreas cancer s), melanoma associated antigens (MART-1, gp 100, MAGE 1,3 tyrosinase), papillomavirus E6 and E7 fragments, whole cells or portions/lysates of antologous tumor cells and allogeneic tumor cells.

An adjuvant may be used to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

A combination therapy including the multivalent protein conjugate and cancer vaccines may have therapeutic synergistic effects on tumors, which would potentially reduce the dosage of the multivalent protein conjugate needed for effective treatment. Thus, side effects associated with non-specific cytotoxicity due to high doses of chemotherapeutic agent can be reduced.

4. Indications for Treatment with the Multivalent Protein Conjugate

Preferable indications that may be treated using the multivalent protein conjugate of the present invention include those involving undesirable or uncontrolled cell proliferation. Such indications include restenosis (e.g. coronary, carotid, and cerebral lesions), benign tumors, a various types of cancers such as primary tumors and tumor metastasis, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a melignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primerary and secondary. Primary tumors arise directly from the tissue in which they are found. A secondary tumor, or metastasis, is a tumor which originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Treatment of abnormal cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

Abnormal angiogenesis that may be may be treated using this invention include those abnormal angiogenesis accompanying rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanim of various angiogenic factors, including elements of the inflammatory response, such as leukocytes, platelets, cytokines, and cicosanoids, or unidentified plasma constituents have yet to be revealed.

In another embodiment of the present invention, a method is provided for treating diseases associated with undesired and uncontrolled angiogenesis. The method comprises administering to a patient suffering from uncontrolled angiogenesis a therapeutically effective amount of a multivalent protein conjugate, such that formation of blood vessels is inhibited. The particular dosage of the multivalent protein conjugate requires to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases.

According to this embodiment, the multivalent protein conjugate may be used to treat a variety of diseases associated with uncontrolled angiogenesis such as retinal/choroidal neovascularization and corneal neovascularization. Examples of retinal/choroidal neovascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disese, systemic lupus erythematosis, retinopathy of prematurity, Eales disease, diabetic retinopathy, macular degeneration,, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

In yet another embodiment of the present invention, a method is provided for treating chronic inflammatory diseases associated with uncontrolled angiogenesis. The method comprises administering a multivalent protein conjugate to a patient suffering from a chronic inflammatory disease associated with uncontrolled angiogenesis a therapeutically effective amount of the multivalent protein conjugate, such that formation of blood vessels is inhibited. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis using the multivalent protein conjugate alone or in conjunction with other anti-inflammatory agents may prevent the formation of the granulosmas, thereby alleviating the disease. Examples of chronic inflammatory disease include, but are not limited to, inflammatory bowel diseases such as Crohn's disease and ulcerative colitis, psoriasis, sarcoidois, and rheumatoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea. These inflammatory bowel diseases are generally caused by chronic granulomatous inflammation throughout the gastrointestinal tract, involving new capillary sprouts surrounded by a cylinder of inflammatory cells. Inhibition of angiogenesis by the multivalent protein conjugate should inhibit the formation of the sprouts and prevent the formation of granulomas. The inflammatory bowel diseases also exhibit extra intestinal manifectations, such as skin lesions. Such lesions are characterized by inflammation and angiogenesis and can occur at many sites other the gastrointestinal tract. Inhibition of angiogenesis by the multivalent protein conjugate should reduce the influx of inflammatory cells and prevent the lesion formation.

Sarcoidois, another chronic inflammatory disease, is characterized as a multisystem granulomatous disorder. The granulomas of this disease can form anywhere in the body and, thus, the symptoms depend on the site of the granulomas and whether the disease is active. The granulomas are created by the angiogenic capillary sprouts providing a constant supply of inflammatory cells. By using the multivalent protein conjugate to inhibit angionesis, such granulomas formation can be inhibited. Psoriasis, also a chronic and recurrent inflammatory disease, is characterized by papules and plaques of various sizes. Treatment using the multivalent protein conjugate alone or in conjunction with other anti-inflammatory agents should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the multivalent protein conjugate alone or in conjunction with other anti-RA agents should prevent the formation of new blood vessels necessary to maintain the chronic inflammation and provide the RA patient relief from the symptoms.

The multivalent protein conjugate may also be used in conjunction with other anti-angiogenesis agents to inhibit undesirable and uncontrolled angiogenesis. Examples of anti-angiogenesis agents include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, matrix metalloproteinase-2 and matrix metalloproteinase-9, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic` acid (LACA), cishydroxyproline, d,1-3,4-dehydroproline, thiaproline], α, α-dipyridyl, β-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), β-1-anticollagenase-serum, α-2-antiplasmin, bisantrene, lobenzarit disodium, n-(2-carboxyphenyl-4-chloroanthronilic acid disodium or “CCA”, thalidomide; angostatic steroid, cargboxynaminolmidazole; metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

5. Compositions, Formulations, and Kits

In a combination therapy involving both a multivalent protein conjugate and another therapeutic agent, the combination preferably has a therapeutic synergy in the treatment of a disease, or a synergistic effect on the subjected being treated. As used herein, a synergistic effect is achieved when a greater therapeutic effect results with a combination therapy than using either drug or monotherapy alone. One advantage of combination therapy with a synergistic effect is that lower dosages of one or both of the drugs or therapies may be used so that the therapeutic index is increased and toxic side effects are reduced.

In an aspect, the invention is directed to kits for treating diseases associated with abnormal cell proliferation and/or angiogenesis. In one embodiment, the kit comprises a container that contains a multivalent protein conjugate; and one or more agents selected from the group conisting of alkylating agent, antibiotic agent, antimetabolic agent, hormonal agent, plant-derived agent, anti-angiogenesis agent and biologic agent.

6. Routes of Administration

A wide variety of delivery methods and formulations for different delivery methods are intended to be encompassed by the therapy of the present invention.

The inventive composition may be administered as compositions that comprise a multivalent protein conjugate or the combination of the conjugate with other therapeutic agents. Such compositions may include, in addition to the inventive combination of therapeutic agents, conventional pharmaceutical excipients, and other conventional, pharmaceutically inactive agents. Additionally, the compositions may include active agents in addition to the inventive combination of therapeutic agents. These additional active agents may include additional compounds according to the invention, or one or more other pharmaceutically active agents. In preferable embodiments, the inventive compositions will contain the active agents, including the inventive combination of therapeutic agents, in an amount effective to treat an indication of interest.

The inventive combination of therapeutic agents and/or compositions may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The inventive combination of therapeutic agents and/or compositions may be administered by a variety of routes, and may be administered or coadministered in any conventional dosage form. Coadministration in the context of this invention is defined to mean the administration of more than one therapeutic in the course of a coordinated treatment to achieve an improved clinical outcome. Such coadministration may also be coextensive, that is, occurring during overlapping periods of time.

One therapeutically interesting route of administration or coadministration is local delivery. Local delivery of inhibitory amounts of inventive combination of therapeutic agents and/or compositions can be by a variety of techniques and structures that administer the inventive combination of therapeutic agents and/or compositions at or near a desired site. Examples of local delivery techniques and structures are not intended to be limiting but rather as illustrative of the techniques and structures available. Examples include local delivery catheters, site specific carriers, implants, direct injection, or direct applications.

Local delivery by a catheter allows the administration of a inventive combination of therapeutic agents and/or compositions directly to the desired site. Examples of local delivery using a balloon catheter are described in EP 383 492 A2 and U.S. Pat. No. 4,636,195 to Wolinsky. Additional examples of local, catheter-based techniques and structures are disclosed in U.S. Pat. No. 5,049,132 to Shaffer et al. and U.S. Pat. No. 5,286,254 to Shapland et al.

Generally, the catheter must be placed such that the inventive combination of therapeutic agents and/or compositions can be delivered at or near the desired site. Dosages delivered through the catheter can vary, according to determinations made by one of skill, but often are in amounts effective to create a cytotoxic or cytostatic effect at the desired site. Preferably, these total amounts are less than the total amounts for systemic administration of the inventive combination of therapeutic agents and/or compositions, and are less than the maximum tolerated dose. The inventive combination of therapeutic agents s and/or compositions delivered through catheters preferably should be formulated to a viscosity that enables delivery through a small treatment catheter, and may be formulated with pharmaceutically acceptable additional ingredients (active and inactive).

Local delivery by an implant describes the placement of a matrix that contains the inventive combination of therapeutic agents s and/or compositions into the desired site. The implant may be deposited by surgery or other means. The implanted matrix releases the inventive combination of therapeutic agents s and/or compositions by diffusion, chemical reaction, solvent activators, or other equivalent mechanisms. Examples are set forth in Lange, Science 249:1527-1533 (September, 1990). Often the implants may be in a form that releases the inventive combination of therapeutic agents s and/or compositions over time; these implants are termed time-release implants. The material of construction for the implants will vary according to the nature of the implant and the specific use to which it will be put. For example, biostable implants may have a rigid or semi-rigid support structure, with the delivery of the inventive composition taking place through a coating or a porous support structure. Other implants made be made of a liquid that stiffens after being implanted or may be made of a gel. The amounts of inventive composition present in or on the implant may be in an amount effective to treat cell proliferation generally, or a specific proliferation indication, such as the indications discussed herein.

One example of local delivery of the inventive composition by an implant is use of a biostable or bioabsorbable plug or patch or similar geometry that can deliver the inventive combination of therapeutic agents and/or composition once placed in or near the desired site. An example of such implants can be found in U.S. Pat. No. 5,429,634 to Narciso, Jr.

A particular application of use of an implant according to the invention is treatment of cell proliferation in tissue that is not highly vascularized, as discussed briefly above. An example of such tissue is bone tissue. The difficulty in treating uncontrolled proliferative cell growth in bone tissue may be exemplified by the difficulties in treating bone tumors. Such tumors are typically refractory to treatment, in part because bone tissue is not highly vascularized. An implant in or near the proliferative site may potentially have localized cytotoxic or cytostatic effects with regard to the proliferative site. Therefore, in one embodiment, the invention may be used to treat bone tumors.

Another example of local delivery by an implant is the use of a stent. Stents are designed to mechanically prevent the collapse and reocclusion of the coronary arteries. Incorporating an inventive combination of therapeutic agents and/or composition into the stent may deliver the agent directly to or near the proliferative site. Certain aspects of local delivery by such techniques and structures are described in Kohn, Pharmaceutical Technology (October, 1990). Stents may be coated with the inventive combination of therapeutic agents and/or composition to be delivered. Examples of such techniques and structures may be found in U.S. Pat. No. 5,464,650 to Berg et al., U.S. Pat. No. 5,545,208 to Wolff et al., U.S. Pat. No. 5,649,977 to Campbell, U.S. Pat. No. 5,679,400 to Tuch, EP 0 716 836 to Tartaglia et al. Alternatively, the inventive combination of therapeutic agents and/or composition loaded stent may be biorotable, i.e. designed to dissolve, thus releasing the inventive combination of therapeutic agents and/or composition in or near the desired site, as disclosed in U.S. Pat. No. 5,527,337 to Stack et al. The present invention can be used with a wide variety of stent configurations, including, but not limited to shape memory alloy stents, expandable stents, and stents formed in situ.

Amounts of the inventive composition delivered by the stent can vary, according to determinations made by one of skill, but preferably are in amounts effective to create a cytotoxic or cytostatic effect at the desired site. Preferably, these total amounts are less than the total amounts for systemic administration of the inventive composition, and are preferably less than the maximum tolerated dose. Appropriate release times can vary, but preferably should last from about 1 hour to about 6 months, most preferably from about 1 week to about 4 weeks. Formulations including the inventive combination of therapeutic agents and/or composition for delivery of the agent via the stent can vary, as determinable by one of skill, according to the particular situation, and as generally taught herein.

Another example is a delivery system in which a polymer that contains the inventive composition is injected into the target cells in liquid form. The polymer then cures to form the implant in situ. One variation of this technique and structure is described in WO 90/03768 to Donn.

Another example is the delivery of the inventive combination of therapeutic agents and/or composition by polymeric endoluminal sealing. This technique and structure uses a catheter to apply a polymeric implant to the interior surface of the lumen. The inventive composition incorporated into the biodegradable polymer implant is thereby released at the desired site. One example of this technique and structure is described in WO 90/01969 to Schindler.

Another example of local delivery by an implant is by direct injection of vesicles or microparticulates into the desired site. These microparticulates may comprise substances such as proteins, lipids, carbohydrates or synthetic polymers. These microparticulates have the inventive composition incorporated throughout the microparticle or over the microparticle as a coating. Examples of delivery systems incorporating microparticulates are described in Lange, Science, 249:1527-1533 (September, 1990) and Mathiowitz, et al., J. App. Poly Sci. 26:809 (1981).

Local delivery by site specific carriers describes attaching the inventive combination of therapeutic agents and/or composition to a carrier which will direct the drug to the desired site. Examples of this delivery technique and structure include the use of carriers such as a protein ligand or a monoclonal antibody. Certain aspects of these techniques and structures are described in Lange, Science 249:1527-1533.

Local delivery also includes the use of topical applications. An example of a local delivery by topical application is applying the inventive combination of therapeutic agents and/or composition directly to an arterial bypass graft during a surgical procedure. Other equivalent examples will no doubt occur to one of skill in the art.

### EXAMPLE

Embodiments of the multivalent protein conjugates (MVPs) of the present invention are constructed and tested for biological functions according to the following protocol.

1. Construction of Expression Vectors of MVPs

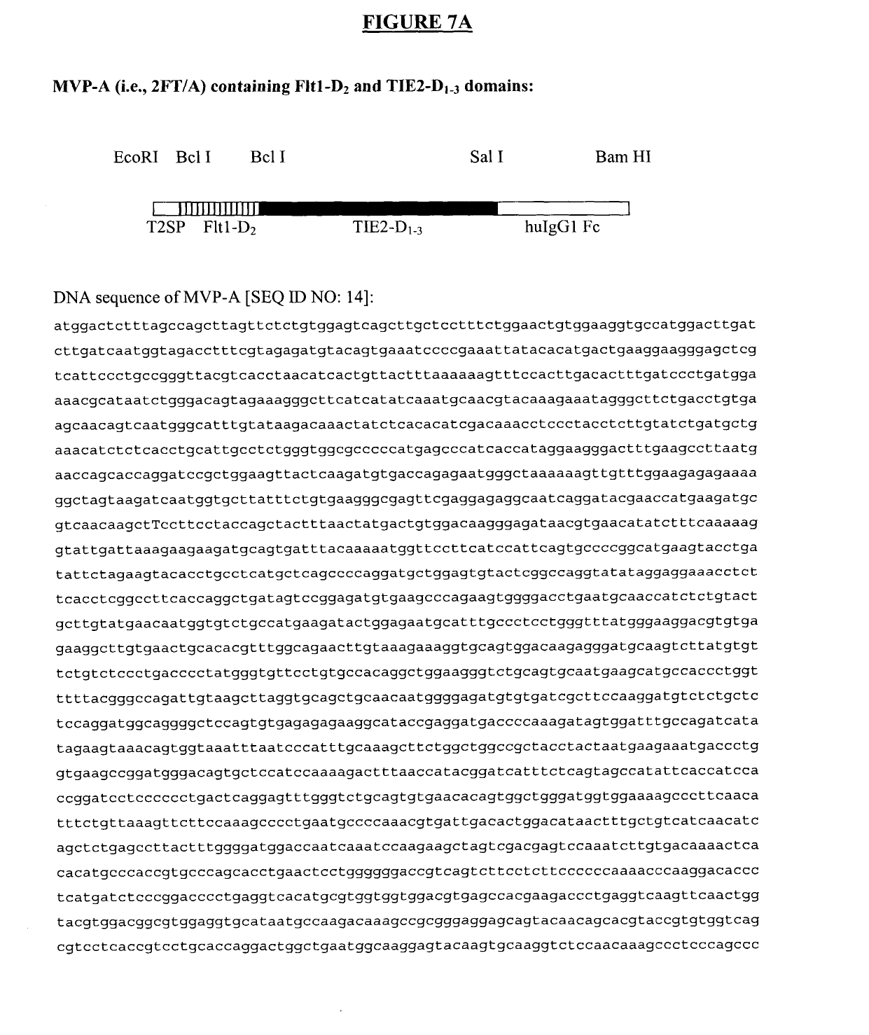

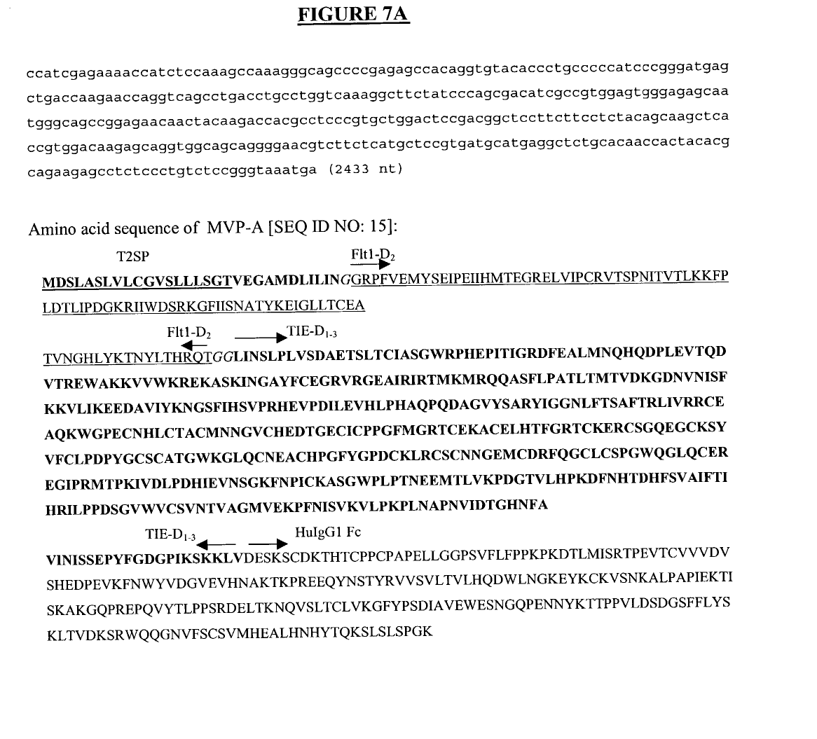

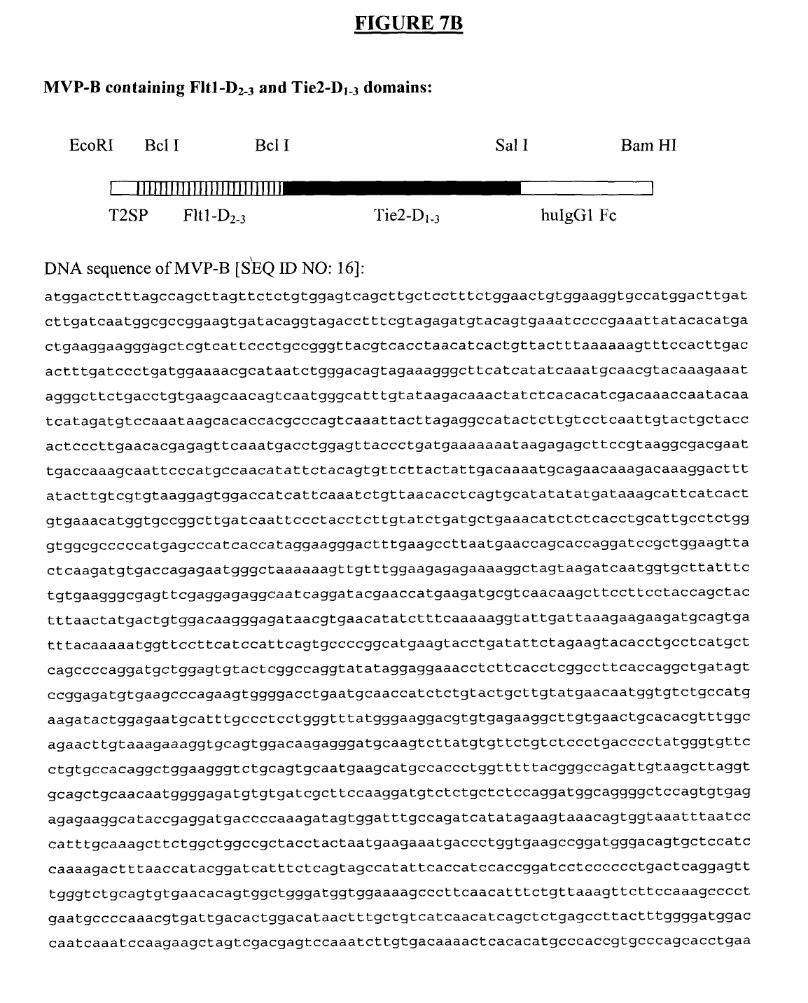

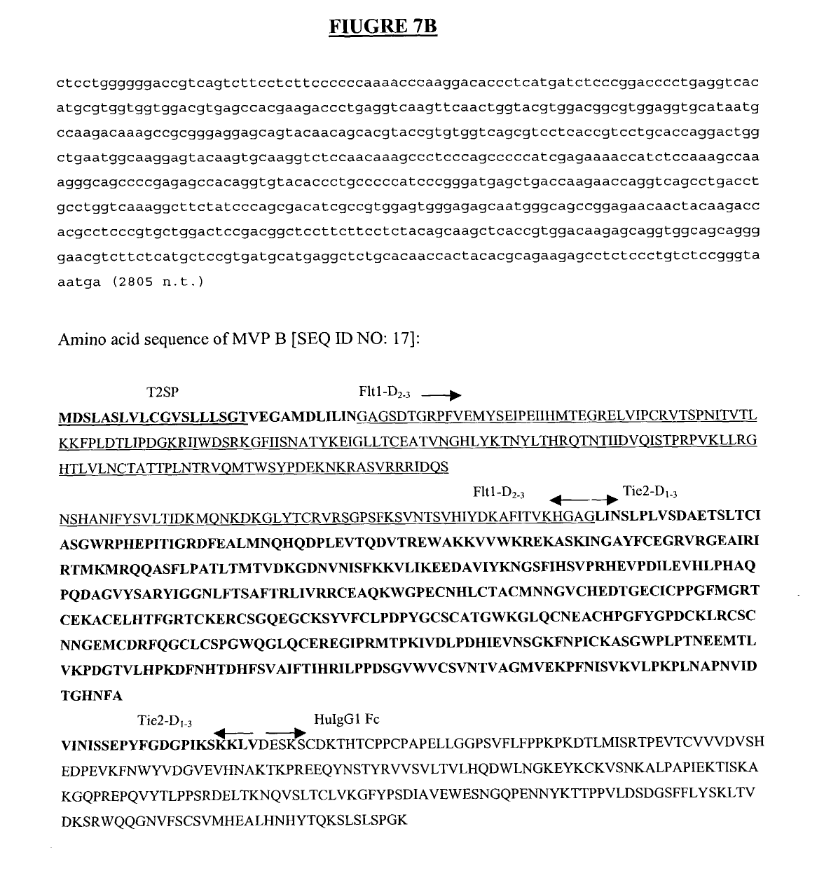

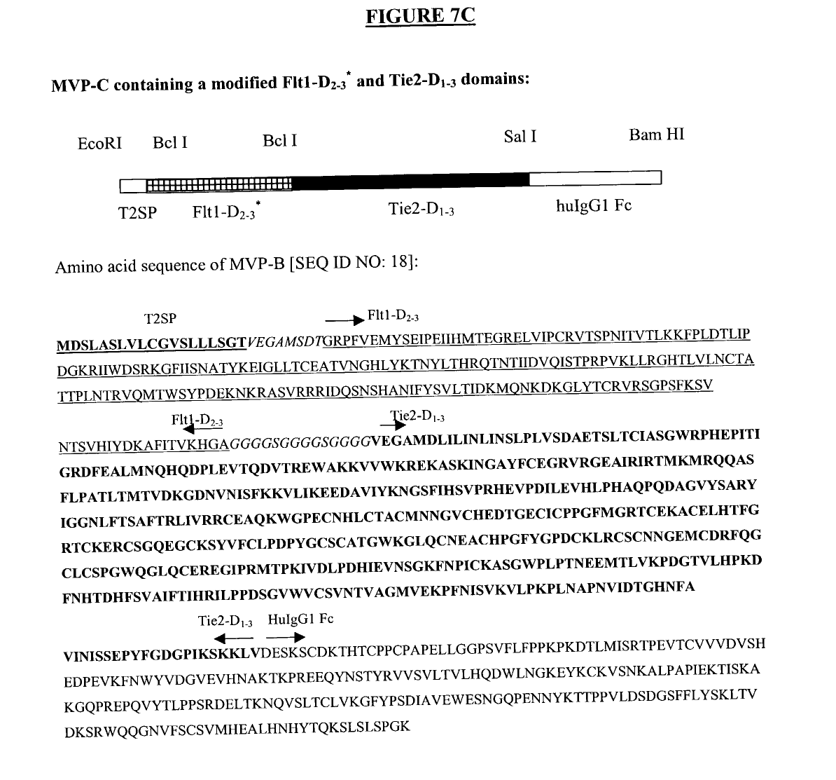

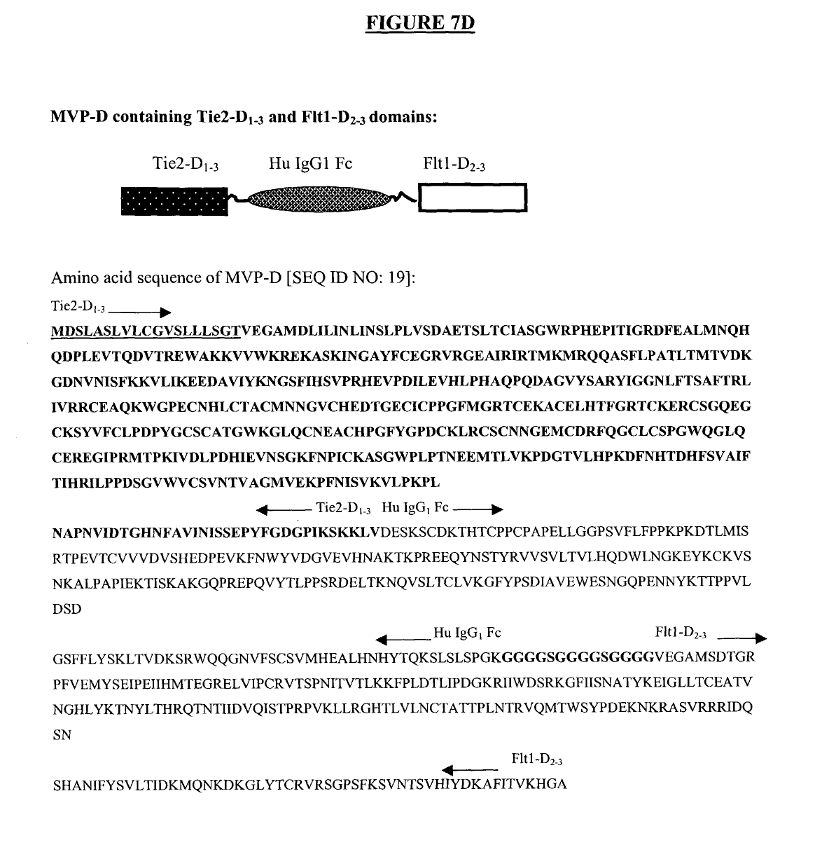

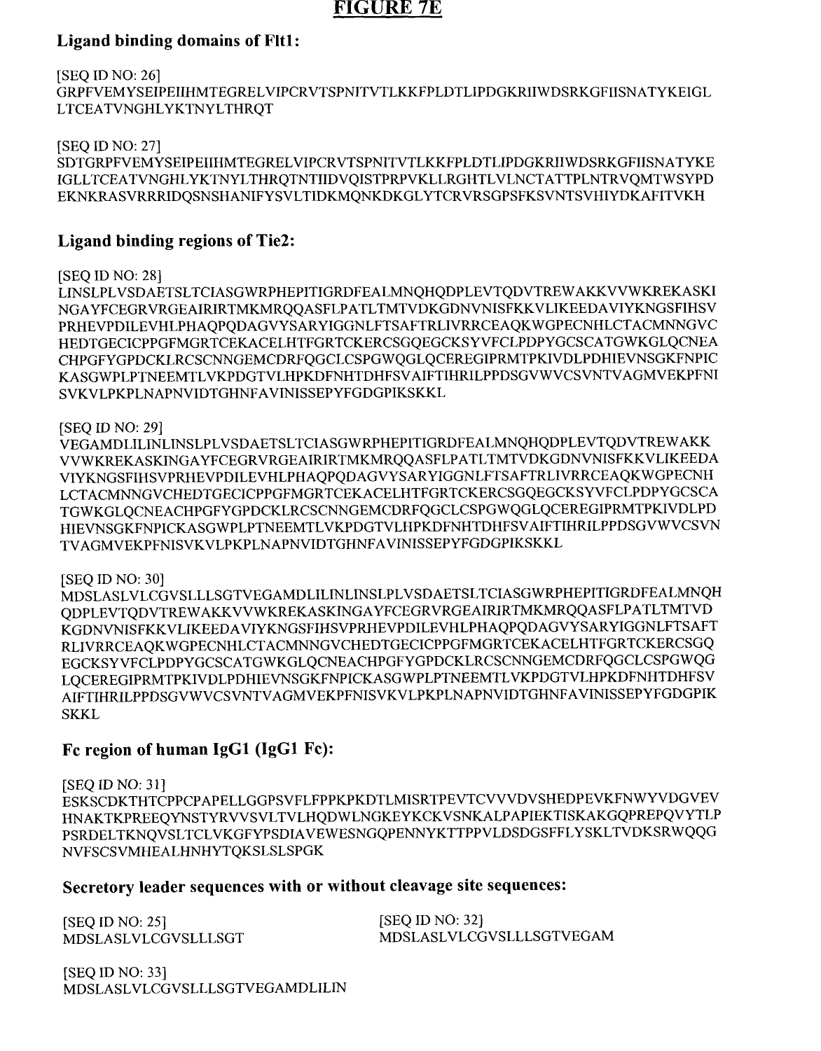

As illustrated in FIG. 7A, one embodiment of the MVP is MVP-A that includes a fragment containing the domain 2 of human VEGF receptor 1, Flt1 -D₂, a fragment containing the extracellular domain (domains 1-3) of the human receptor for angiopoietin 1 (Tie2/TEK), Tie2-D₁₋₃, and the constant region (Fc) of human IgG1 as a tag. In another embodiment, as illustrated in FIG. 7B, MVP-B includes a fragment containing domain 2 and 3 of VEGF receptor 1, Flt1-D₂₋₃, a fragment containing Tie2-D₁₋₃, and the human IgG1 Fe as a tag.

The DNA fragment encoding the extracellular domain (ECD) of Tie2/TEK (labeled as Tie2-D₁₋₃ in FIGS. 7A-B, 742 amino acid residues including the signal peptide) was amplified from human fetal spleen cDNA by polymerase chain reaction (PCR) using pfu polymease and a forward prime:

5′-ATGAATTCATGGACTCTTTAGCCAGCTTAGTTCTC-3′ [SEQ ID NO: 5] and a reverse primer:

5′-ATGTCGACGAGGTCCGCTGGTGCTTGAGA-3′ [SEQ ID NO: 6].

A 2.24 kb DNA fragment was amplified under this thermocycling condition: 94° C., 1 min→52° C., 0.5 min→72° C., 3.0 min for 30 cycles at 0.5 μM prime mix. At the end of cycling, additional 10 min incubation at 72° C. was performed. The PCR product was determined by agarose gel electrophoresis using 0.7% agarose gel. The 2.24 kbp fragment was purified using a PCR purification kit (Qiagen) and cut with EcoRI and Sal I restriction enzyme. The resultant restriction fragment was purified by agarose gel electrophoresis and cloned into the EcoRI/SalI site of the plasmid pCMV-FLAG-3a (Sigma), resulting a plasmid construct pSJ-T2X-5 encoding human Tie2/TEK extracellular domain fused to FLAG.

The FLAG tag on the plasmid construct pSJ-T2X-5 was replaced with human IgG1 Fc fragment that was amplified from the same human fetal spleen cDNA sample by PCR using a forward primer:

5′CTA GTC GAC GAG TCC AAA TCT TGT GAC AAA ACT-3′ [SEQ ID NO: 7]

and a reverse primer:

5′TCC CTG TCT CCG GGT AAA TGA GGA TCC GGT GGT ACC GAT3′ [SEQ ID NO: 8]

A DNA fragment of 723 bp was prepared, purified and treated with restriction endonuclease, Sal I and Kpn I. The treated fragment was ligated into pSJ-T2X-5 to obtain a new plasmid, pSD-T2-Fc, encoding Tie2/TEK extracellular domain fused to human IgG1 Fc fragment.

To make a DNA fragment coding Tie2/TEK D₁₋₃ from pSD-T2-Fc, a deletion of its fibronection type III domain was conducted by PCR using pSD-T2-Fc as a template, primer [SEQ ID NO: 7] as a forward primer and a reverse primer:

5′CCAATCAAATCCAAGAAGCTAGTCGACGAGTCCAA3′ [SEQ ID NO: 9].

The PCR mix containing the amplicon was directly transferred into E. coli cells and the resulting plasmid, pSD-Tie2/TEK-D₁₋₃ (1442 base pairs) (also encoding human IgG1 Fc) was obtained. The DNA sequence of the inserts was confirmed by dideoxylnucleotide chain termination reaction.

The DNA fragments encoding portions of Flt1 containing the second Ig-like domain of Flt1, Flt1-D₂ (132-227 a.a. residues) were amplified from human fetal brain cDNA (Invitrogen A3 10047) by PCR using pfu polymerase and a forward primer:

5′-TTG ATC TTG ATC AAT GGC GGT AGA CCT TTC GTA GAG ATG-3′ [SEQ ID NO: 10]

and a reverse primer:

5′-GGA ATT GAT CAA ACC GCC GGT TTG TCG ATG TGT GAG ATAG-3′ [SEQ ID NO: 11]

A DNA fragment encoding the protein sequence containing the second and the third Ig-like domains of human flt 1 extracellular domain, Flt1-D₂₋₃ (129-338 a.a. residues), was amplified by PCR from the same cDNA sample using the following primer pair:

The forward primer:

5′TGG ACT TGA TCT TGA TCA ATG GCG CCG GAA GTG ATA CAG GTA GAC CTT TC3′ [SEQ ID NO: 12]

The reverse primer:

5′GCA TTC ATC ACT GTG AAA CAT GGT GCC GGC TTG ATC AAT TCC CTA CCT C3′ [SEQ ID NO: 13].

A Bcl I restriction site was included in each primer in order to insert the Flt1-D₂ or Flt1D₂₋₃ into the N-terminal region, 11 amino acid residues behind Tie2/TEK signal peptide sequence. The PCR mix (50 μl) contained 0.2 μM primer mix, 0.25 mM dNTP(dCTP, dATP, dGTP, dTTP), 2 μl human fetal brain cDNA and 1 μl pfu enzyme. The thermocycling condition was setup as follows: 94° C., 30s→52° C., 30s→72° C., 45s for 25 cycles and at the end of cycling, additional 10 minute incubation at 72° C. was performed. The PCR product was determined by agarose gel electrophoresis using 1.5% agarose. The DNA fragments encoding Flt1-D₂ (288 bp) and Flt1D₂₋₃ (627 bp) were purified using PCR purification kits (Qiagen).

The purified Flt1-D₂ or Flt1D₂₋₃ fragment was treated with Bcl I and inserted into pSD-Tie2/TEK-D₁₋₃. After ligation, the new constructs were transformed into DH5α competent cells and colonies containing Flt1-D₂ or Flt1-D₂₋₃ were selected and confirmed by PCR and DNA sequencing. The plasmid DNA encoding a multivalent binding protein, Flt1-D₂-Tie2-D₁₋₃-Fc (FIG. 7A, DNA sequence [SEQ ID NO: 14] and amino acid sequence [SEQ ID NO: 15]) or Flt1-D₂₋₃-GG-Tie2-D₁₋₃-Fc (FIG. 7B, DNA sequence [SEQ ID NO: 16] and amino acid sequence [SEQ ID NO: 17]), with correct sequence was prepared and used for transfection of COS-7 cells.

Alternative designs of MVPscontaining ligand-binding domains of Flt1 and Tie2, are shown in FIGS. 7C and 7D. As shown in FIG. 7C, MVP contains a modified Flt1-D₂₋₃ at the N-terminus, followed by Tie2-D₁₋₃ with human IgG1 Fc fused to the C-terminus of Tie2-D₁₋₃. Also shown is the amino acid sequence of MVP-C [SEQ ID NO: 18]. As shown in FIG. 7D, MVP-D contains Tie2-D₁₋₃, followed a modified Flt1-D₂₋₃ at the N-terminus followed by a modified Flt1-D₂₋₃ at the N-terminus. In MVP-D human IgG1 Fc is fused to the C-terminus of Tie2-D₁₋₃ and linked to the N-terminus of the modified Flt1-D₂₋₃ via a GGGGSGGGGSGGGG linker [SEQ ID NO: 20]. Also shown is the amino acid sequence of MVP-D [SEQ ID NO: 19].

Another plasmid for expressing the MVPs of the present invention was also constructed. A Dhfr (dihydrofolate reductase) cassette was incorporated into the plasmid constructed above and the Kozak sequence was added to the upstream of the start codon for MVP translation.

The Dhfr cassette (1,277 bp) was amplified by PCR with Pfu DNA polymerase using the murine beta-globin transcriptional regulation unit and the Mus Musculus Dhfr gene as a template. Both forward and reverse primers contained a Spe I restriction site.

Forward: TGTTGACATTGAGCTGGGACTAGTAGCTTTG [SEQ ID NO: 21]

Reverse: CCGTAATTGATTAAGAATGACAACTAGTCAGACAATG). [SEQ ID NO: 22]

The resulting amplicon was digested by Spe I as the original vector contains a unique Spe I site before the poly-linker region. The amplicon was inserted into the vector by ligation.

The incorporation of the Kozak sequence into the upstream of the 2FT/A cDNA was performed as follows. The Kozak sequence was PCR amplified by the Pfu enzyme using the original vector (Amplicon size is 870 bp). The forward primer contained an Eco RI restriction site and the Kozak sequence upstream of the initiation codon.

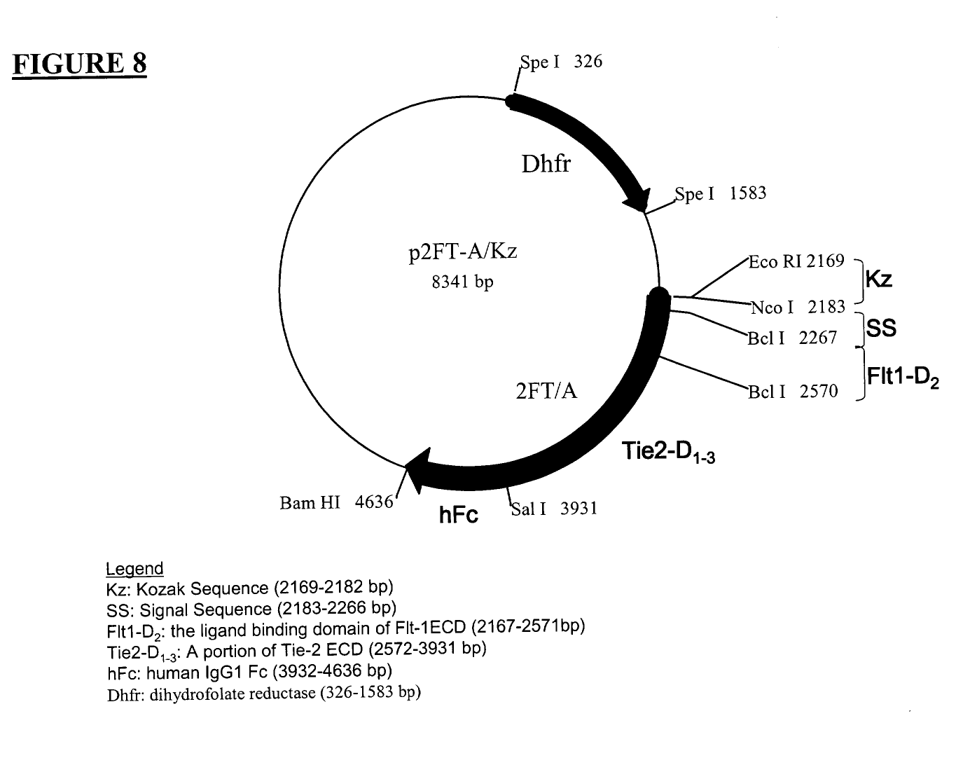

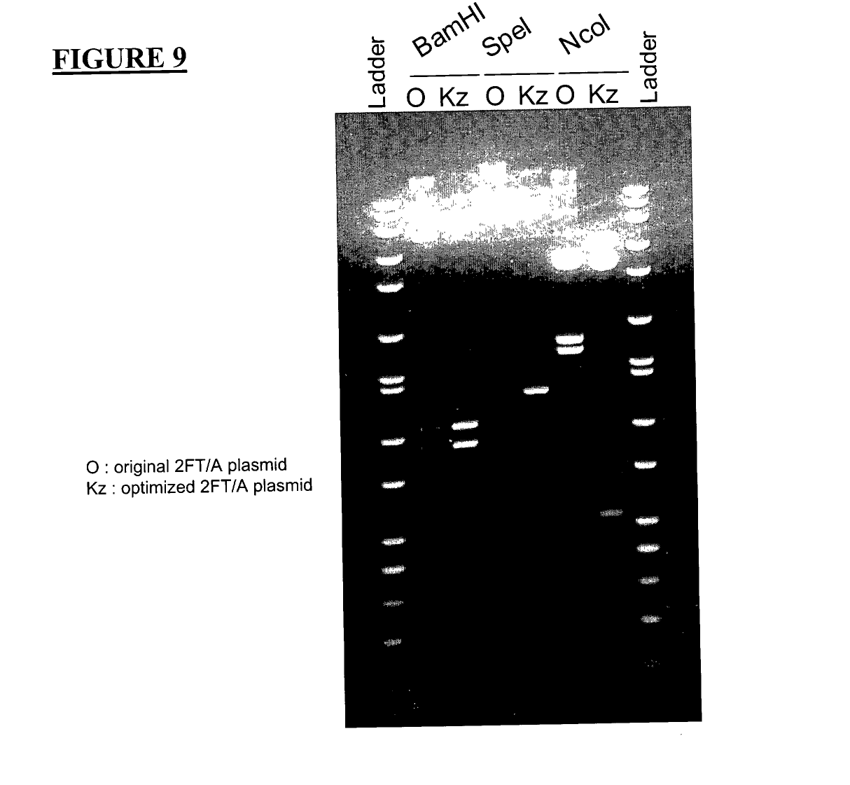

(GTTTAGGAATTCGTCAGCCACC [symbol] GACTCTTTAG, [SEQ ID NO: 23), while the reverse primer (TGAGCATGAGGCAGGTGTAC, SEQ ID NO: 24) was located in the area encoding the Tie2 portion of the MVP-A, after the Xba I restriction site. The amplicon and the intermediate form of the vector containing the Dhfr cassette were digested by Eco RI and Xba I and ligated together, resulting a plasmid designated as p2FT/A-Dhfr/Kz that encodes MVP-A (thereafter referred to as 2FT/A). FIG. 8 shows the restriction map of the plasmid p2FT/A-Dhfr/Kz. About 250 ng of DNA of p2FT/A-Dhfr/Kz was digested with Bam HI, Spe I and Nco I in three separate reactions. FIG. 9 shows the restriction mapping the p2FT/A-Dhfr/Kz via agarose gel electrophoresis and compares its pattern with that of the original plasmid without the Dhfr cassette and the Kozak sequence. The restriction mapping indicates that p2FT/A-Dhfr/Kz was successfully constructed. Expression vectors of other MVPs, such as MVP-B, -C and -D, are constructed following protocols similar to what is described above for the plasmids encoding MVP-A (or 2FT/A).

2. Expression, Purification and Characterization of MVPs

Monolayer cultures (90% confluence) of COS-7 cells were transfected with the plasmid DNA encoding a MVP constructed above at 16 μg/flask (Falcon T150) and grown in DMEM containing 20% fetal bovine serum at 37° C. for 36 hrs and the culture temperature was shifted to 32° C. for another 36 hrs before harvesting. The cell culture supernatant was filtered through a 0.2μ filter and the cell culture supernatant was referred to as harvested cell culture fluid (HCCF).

The multivalent protein conjugates (MVPs) constructed above were purified using a protein A Sepharose 4B column or ProSep A (Millipore) and Q-Sepahrose fast flow column chromatography and analyzed by SDS-PAGE. For example, 2FT/A was eluted from the column with 0.1 M acetate buffer (pH 2.9) and neutralized immediately with 2 M Tris base to pH 7.0. The preparation was concentrated with 40% saturated ammonium sulfate (NH₄)₂SO₄ for 30 min, precipitated by centrifugation at 4000 rpm (Beckman rotor type JS 4.2) for 30 min and the pellet was dialyzed against PBS at 4° C. for 12 hr with 5 changes of PBS. The 2FT/A was clarified by centrifugation at 10,000×g for 10 min and supernatant was collected and further assayed for protein concentration using BCA method (Pierce). The purity was analyzed by SDS-PAGE.

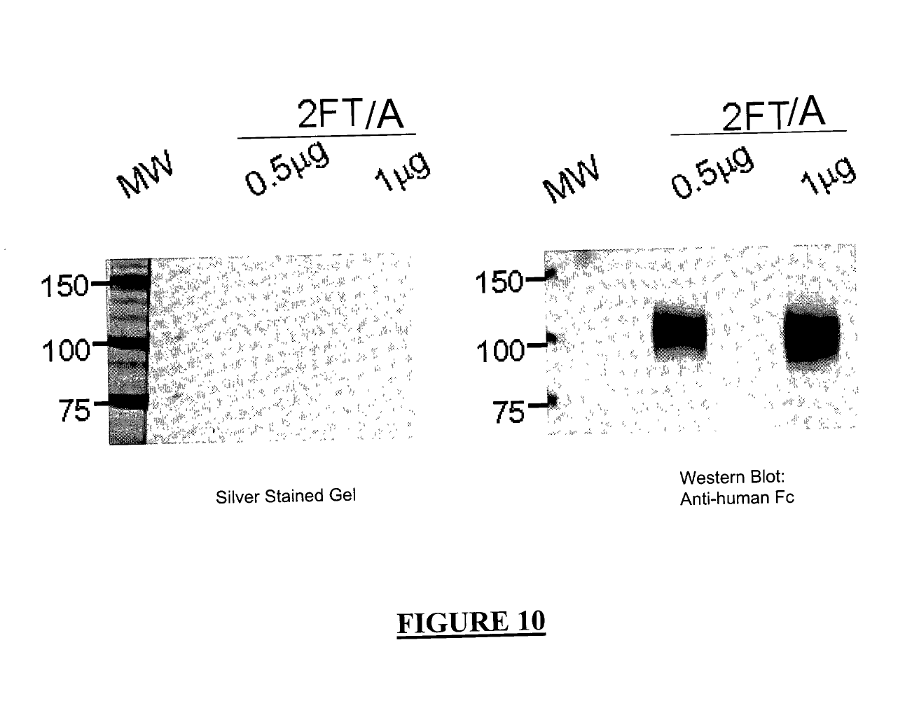

FIG. 10 shows SDS-PAGE analysis of 2FT/A expressed by p2FT/A-Dhfr/Kz. Briefly, two samples of purified MVP-A of 0.5 and 1 μg in duplicate were loaded on an 8-16% acrylamide gradient gel. Half of the gel was subjected to silver staining (Pierce) (FIG. 10, left panel); the remaining half was transferred onto a PVDF membrane (Millipore, Bedford) and was subjected to Western blotting with an AP-conjugated anti-human Fc antibody (Rockland). Specific antigen-antibody interaction between HuIgG1 Fc and the anti-human Fc antibody was revealed by incubation with BCIP/NBT reagent (Pierce) for 5 min. Two main bands were detected both by silver staining and western blotting, at ˜100 kDa (corresponding to the expected molecular weight of the 2FT/A protein) and ˜50 kDa (corresponding to Fc fragment generated by partial degradation of the 2FT/A protein).

3. Functional Analysis of MVPs

a) Binding of VEGF and Angiopoietin-1 to 2FT/A

Binding of 2FT/A that contains Flt1-D₂ and Tie2-D₁₋₃ to the cognate ligand of Flt1, human VEGF, was analyzed by an ELISA binding assay. Approximately 2FT/A at 10 μg/ml was coated onto a 96-well microplate at 25 μl/well in 0.1 M carbonate buffer (pH 9.6) and incubated at 4° C. overnight. The plate was blocked with 3% milk PBS-T at 37° C. for 60 min. Human VEGF (Calbiochem, La Jolla, Calif.) at various concentrations, 0, 0.1. 0.25, 0.5, 1, 2.5, 5, and 10 μg/ml, was added to the plate and incubated at 37° C. for 60 min. The bound VEGF was probed with a myc-tagged anti-VEGF binding antibody and incubated at 37° C. for 60 min. The bound anti-VEGF-myc was detected with a mouse HRP conjugate of an anti-myc antibody.

b) Inhibition of Endothelial Cell Growth by 2FT/A

Functional assessment for 2FT/A was performed for its ability to inhibit endothelial cell proliferation stimulated by VEGF (Calbiochem, La Jolla). Briefly, from a subconfluent mono-layer of bovine brain capillary endothelial (BBE) cells, 12,500 cells were plated in 0.5 ml in a 24-well plate using a growth medium containing 10% calf serum (CS). After 24 hours, the growth medium was changed to 0.5% CS medium at 0.5 ml/well. After 18 hours of serum starvation, stimulating factors were added for 20 hrs before pulsing cells with the MTT (3-[4,5-Dimethylthiazol-2-yl]-2,5-diphenyltetrazolium bromide). MTT was added at 1/10 final dilution (5 mg/ml stock solution) to each well and incubated for 3 hours in incubator. At the end of the incubation period, the medium was removed. The converted dye was solubilized with acidic isopropanol at 0.25 ml per well. Half of the solubilized precipitate was transferred into a 96-well plate and the absorbance measured at a wavelength of 570 nm with background subtraction at 660 nm.

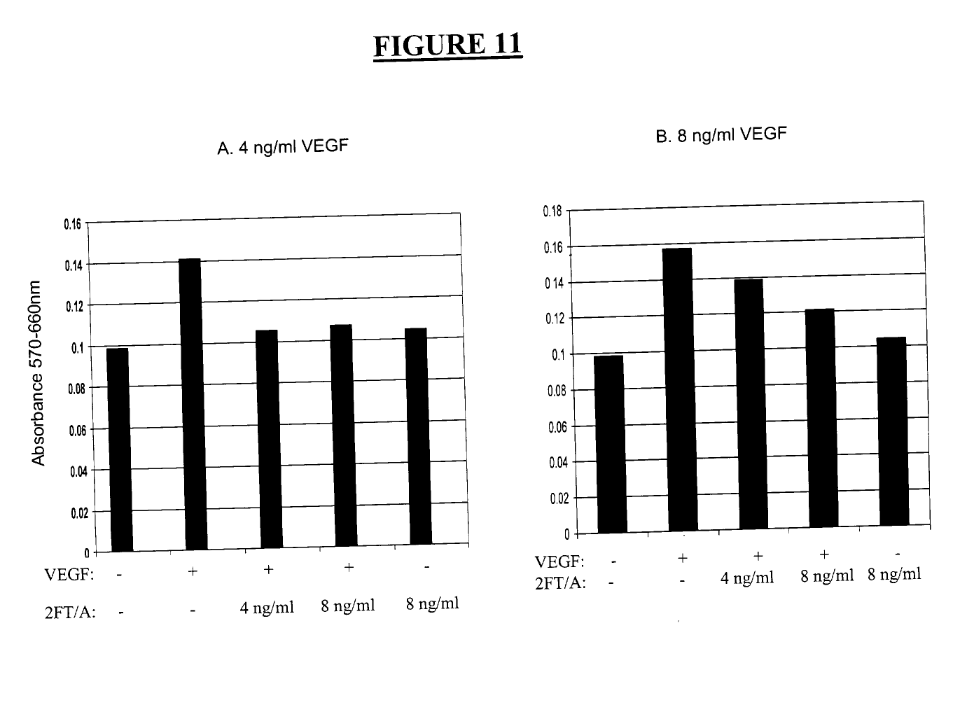

The results from the MTT assay are shown in FIG. 11. As shown in FIG. 11, 2FT/A blocked the VEGF stimulated endothelial cell proliferation at 4 ng/ml (shown in the left panel) and 8 ng/ml (shown in the right panel) in vitro. The inhibitory effect is also dose-dependent as shown in the right panel of FIG. 11. 2FT/A alone did not show any toxicity in the cell culture. These results demonstrate that the ligand binding domain of Flt1 (Flt1-D₂) fused to that of Tie2 (Tie2-D₁₋₃) can still exert its biological function by inhibiting VEGF-stimulated cell proliferation in vitro, which is comparable to the activity of an unfused Flt1-D₂ demonstrated in the art (Weismann C et al. (1997) Cell 91:695-704; and Starovasnik MA et al. (1999) J. Mol. Biol. 293:531-544).

c) Inhibition by 2FT/A of bFGF-induced BBE Cell Proliferation via Endocrine Loop

Functional assessment for 2FT/A was performed for its ability to inhibit endothelial cell proliferation stimulated by bFGF (Promega, Madison). Briefly, from a subconfluent mono-layer of bovine brain capillary endothelial (BBE) cells, 12,500 cells were plated in 0.5 ml in a 24-well plate using a growth medium containing 10% calf serum (CS). After 24 hours, the growth medium was changed to 0.5% CS medium at 0.5 ml/well. After 18 hours of serum starvation, stimulating factors were added for 20 hrs before pulsing cells with the MTT (3-[4,5-Dimethylthiazol-2-yl]-2,5-diphenyltetrazolium bromide). MTT was added at 1/10 final dilution (5 mg/ml stock solution) to each well and incubated for 3 hours in incubator. At the end of the incubation period, the medium was removed. The converted dye was solubilized with acidic isopropanol at 0.25 ml per well. Half of the solubilized precipitate was transferred into a 96-well plate and the absorbance measured at a wavelength of 570 nm with background subtraction at 660 nm.

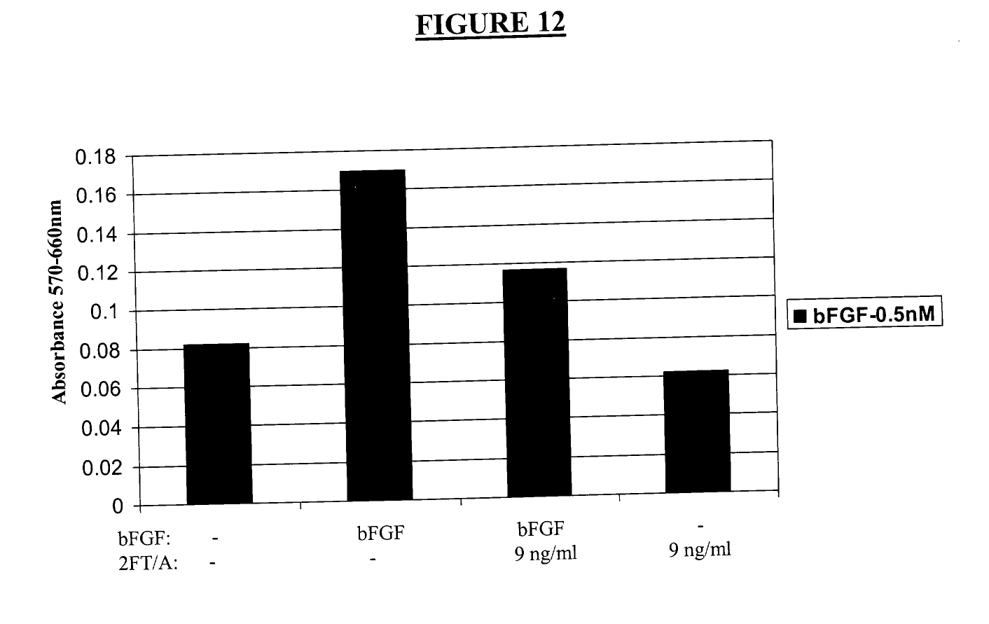

Results from the MTT assay are shown in FIG. 12. As shown in FIG. 12, 2FT/A blocked the bFGF stimulated endothelial cell proliferation at 8 ng/ml (shown in the right panel) in vitro. These data suggest that the bFGF angiogenic effect may be related to the VEGF expression and release in BBE cells. In fact, others have shown that bFGF induces VEGF secretion that stimulates BBE cells proliferation as an autocrine/paracrine loop. Claffey KP et al. (2001) Lab Invest. 81(1):61-75; and Pepper MS et al. (1998) J Cell Physiol 177:439-52. Even though 2FT/A does not contain a binding domain of bFGF receptor, inhibition of the VEGF-induced proliferation of BBE cells is sufficient to abolish the bFGF effect. Our results further demonstrate that the ligand-binding domain of Flt1 (Flt1-D₂) fused to that of Tie2 (Tie2-D₁₋₃) is biologically functional in inhibiting VEGF-stimulated cell proliferation. These results also demonstrate that vascular endothelial growth factor (VEGF) and basic fibroblast growth factor (bFGF) are potent angiogenic inducers that act synergistically in in vitro cell-based assays.

d) Chick Chorioallantoic Membrane (CAM) Assay

The inhibitory effects of the MVPs on angiogenesis induced by VEGF, bFGF and angiopoietin 1 are measured by using a chick chorioallantoic membrane (CAM) assay (Crum et al. (1985) Science 230:1375). Briefly, fertilized chick embryoes are removed from their shell on day 3 and 4, and a methylcellulose disc containing the MVP is implanted on the chorioallantoic membrane. The embryos are examined 48 hours later, and, if a clear avascular zone appears around the methylcellulose disc, the diameter of that zone is measured and compared with those of a positive control (e.g., treatment with thalidomide) and a negative control (without addition of a drug).

e) Basic Fibroblast Growth Factor (bFGF)-induced Corneal Neovascularization

The activity of the MVP in bFGF induced corneal neovascularization is also determined in a rabbit cornea angiogenesis assay. Pellets for implantation into rabbit corneas are made by mixing 110 μl of saline containing 12 μg of recombinant bFGF (Takeda Pharmaceuticals-Japan) with 40 mg of sucralfate (Bukh Meditec-Denmark); this suspension was added to 80 μl of 12% hydron (Interferon Sciences) in ethanol. 10 μl aliquots of this mixture was then pipetted onto teflon pegs and allowed to dry producing approximately 17 pellets. A pellet was implanted into corneal micropockets of each eye of an anesthetized female New Zealand white rabbit, 2 mm from the limbus followed by topical application of erythromycin ointment onto the surface of the cornea. The animals are injected intravenously with the MVP constructed above daily from 2 days post-implantation. The animals are examined with a slit lamp every other day in a masked manner by the same corneal specialist. The area of corneal neovascularization was determined by measuring with a reticule the vessel length (L) from the limbus and the number of clock hours (C) of limbus involved.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A multivalent protein conjugate having a general structural formula:
BD₁—L—(BD)ₙ₋₂—L—BDₙ,
wherein BD is a ligand-binding domain of a receptor, L is a covalent bond or a linker moiety, and n is an integer from two to about fifty.

2. The multivalent protein conjugate of claim 1, wherein BD₁, (BD)ₙ₋₂, and BDₙ is ligand-binding domains from n different receptors.

3. The multivalent protein conjugate of claim 1, wherein BD₁, (BD)ₙ₋₂, and BDₙ is the same ligand binding domain of a receptor.

4. The multivalent protein conjugate of claim 1, wherein n equals three or more, and two or more of BD₁, (BD)ₙ₋₂, and BDₙ are the same ligand-binding domain of a receptor.

5. The multivalent protein conjugate of claim 1, wherein the ligand-binding domain is a ligand-binding domain of a cell surface receptor.

6. The multivalent protein conjugate of claim 5, wherein the cell surface receptor is a cell surface receptor for a growth factor or a G-protein-coupled receptor.

7. The multivalent protein conjugate of claim 1, wherein the ligand-binding domain is a ligand-binding domain of a receptor for a ligand selected from the group consisting of epidermal growth factors, transferrin, insulin-like growth factor, transforming growth factors, interleukin-1, and interleukin-2.

8. The multivalent protein conjugate of claim 1, wherein the ligand-binding domain is a ligand-binding domain of a receptor for an angiogenic factor.

9. The multivalent protein conjugate of claim 8, wherein the receptor for an angiogenic factor is selected from the group consisting of angiostatin-R, angiostadin binding protein I, low-affinity receptors for glypicans, endostatin-R, endothelin-A receptor, angiocidin-R, angiogenin-R, CD36, CD47, and tumstatin-R.

10. The multivalent protein conjugate of claim 1, wherein the ligand binding domain is a ligand-binding domain of a receptor for an angiogenic growth factor.

11. The multivalent protein conjugate of claim 10, wherein the receptor for an angiogenic growth factor is selected from the group consisting of VE-cadherin, Flt1, KDR, Flt4, NP-1, NP-2, Tie1, Tie2, FGF-R1, FGF-R2, FGF-R3, and FGF-R4, PDGF-R, Eph A1-8, and Eph B1-8.

12. The multivalent protein conjugate of claim 1, wherein the ligand-binding domain is a ligand-binding domain of Flt1 comprising SEQ ID NO: 26.

13. The multivalent protein conjugate of claim 1, wherein at least one of the ligand-binding domain BD₁₋ₙ is a ligand-binding domain of Flt1 comprising SEQ ID NO: 27.

14. The multivalent protein conjugate of claim 1, wherein at least one of the ligand-binding domain BD₁₋ₙ is ligand-binding domain of Tie2 comprising SEQ ID NO: 28, 29 or 30.

15. The multivalent protein conjugate of claim 1, wherein n equals 2, and the amino acid sequence of BD₁ comprises SEQ ID NO: 26 or 27 and the amino acid sequence of BD₂ comprises SEQ ID NO: 28, 29, or 30.

16. The multivalent protein conjugate of claim 1, wherein the amino acid sequence of the multivalent protein conjugate comprises a sequence selected from the group consisting of 15, 17, 18, and 19.

17. The multivalent protein conjugate of claim 1, wherein the ligand-binding domain is a ligand-binding domain of a G-protein-coupled receptor.

18. The multivalent protein conjugate of claim 17, wherein the G-protein-coupled receptor is a receptor for sphingosie-1-phosphate or edg receptor.

19. The multivalent protein conjugate of claim 1, wherein the ligand-binding domain is a ligand-binding domain of a cytokine receptor.

20. The multivalent protein conjugate of claim 19, wherein the cytokine receptor is a receptor for tumor necrosis factor-α or interleukin-8.

21. The multivalent protein conjugate of claim 1, wherein the ligand-binding domain is a ligand-binding domain of an integrin.

22. The multivalent protein conjugate of claim 21, wherein the integrin is αvβ3 or α2vβ1 integrin.

23. The multivalent protein conjugate of claim 1, wherein the ligand-binding domain is a ligand-binding domain of a matrix metalloprotease.

24. The multivalent protein conjugate of claim 1, wherein the ligand-binding domain is a ligand-binding domain of a nuclear hormone receptor.

25. The multivalent protein conjugate of claim 24, wherein the nuclear hormone receptor is selected from the group consisting of estrogen, androgen, retinoid, vitamin D, glucoccoticoid and progestrone receptors.

26. The multivalent protein conjugate of claim 1, wherein the linker moiety L is a polypeptide linker.

27. The multivalent protein conjugate of claim 26, wherein the polypeptide linker is selected from the group consisting of Gly-Gly [SEQ ID NO: 1], Gly-Ala-Gly [SEQ ID NO: 2], or Gly-Pro-Ala [SEQ ID NO: 3], and Gly-Gly-Gly-Gly-Ser [SEQ ID NO: 4].

28. The multivalent protein conjugate of claim 26, wherein the polypeptide linker is the constant region of human IgG1, IgG2 or IgG4.

29. The multivalent protein conjugate of claim 26, wherein the polypeptide linker is human IgG1 Fc having an amino acid sequence of SEQ ID NO: 31.

30. The multivalent protein conjugate of claim 1, wherein the linker moiety L is an oligopeptide selected from the group consisting of polyglycine, polyserine, polyproline, and polyalanine.

31. The multivalent protein conjugate of claim 1, further comprising a secretory leader sequence in the N-terminus of any of the ligand-binding domain.

32. The multivalent protein conjugate of claim 31, wherein the secretory leader sequence comprises SEQ ID NO: 25, 32 or 33.

33. The multivalent protein conjugate of claim 1, further comprising an oligomerization unit.

34. The multivalent protein conjugate of claim 33, wherein the oligomerization unit is attached to the N-terminus or the C-terminus of the conjugate.

35. The multivalent protein conjugate of claim 33, wherein the oligomerization unit is positioned between two ligand-binding domains in the conjugate.

36. The multivalent protein conjugate of claim 33, wherein the oligomerization unit is selected from the group consisting of the dimerization unit of receptors for opioid, muscarinic, dopamine, serotonin, adenosine/dopamine, and GABA-B.

37. The multivalent protein conjugate of claim 33, wherein the oligomerization unit is selected from the group consisting of the leucine zipper domain of the nuclear oncoproteins Jun and Fos, and the leucine zipper domain of the proto-oncoproteins Myc and Max.

38. The multivalent protein conjugate of claim 1, further comprising a tag peptide sequence (Tag).

39. The multivalent protein conjugate of claim 38, wherein the Tag is attached to the N-terminus, the C-terminus, or both termini of the conjugate.

40. The multivalent protein conjugate of claim 38, wherein the Tag is selected from the group consisting of the constant region (Fc) of human IgG1, IgG2 or IgG4, a polyhistidine tract, polyarginine, polylysine, glutathione-S-transferase (GST), maltose binding protein, a portion of staphylococcal protein A, FLAG, a myc tag, virus hemoagglutin and various immunoaffinity tags, and an EE tag.

41. The multivalent protein conjugate of claim 40, wherein tag peptide is human IgG1 Fc having an amino acid sequence of SEQ ID NO: 31.

42. A multivalent protein conjugate having a general structural formula selected from the group consisting of:
BD₁—L—Tag—(BD)ₙ₋₂—L—BDₙ,
BD₁—L—(BD)ₙ₋₂—Tag—L—BDₙ,
BD₁—L—Tag—L—(BD)ₙ₋₂—L—BDₙ,
and
Tag—BD₁—L—Tag—L—(BD)ₙ₋₂—L—BDₙ,
wherein BD is a ligand-binding domain of a receptor, L is a covalent bond or a linker moiety, Tag is a tag peptide sequence, and n is an integer from two to about fifty.

43. The multivalent protein conjugate of claim 42, wherein the Tag is selected from the group consisting of the constant region (Fc) of human IgG1, IgG2 or IgG4, a polyhistidine tract, polyarginine, polylysine, glutathione-S-transferase (GST), maltose binding protein, a portion of staphylococcal protein A, FLAG, a myc tag, virus hemoagglutin and various immunoaffinity tags, and an EE tag.

44. The multivalent protein conjugate of claim 42, wherein tag peptide is human IgG1 Fc having an amino acid sequence of SEQ ID NO: 31.

45. A multivalent protein conjugate having a general structural formula: 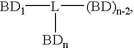
wherein BD is a ligand-binding domain of a receptor, L is a branched linker moiety, and n is an integer from three to about fifty.

46. The multivalent protein conjugate of claim 45, wherein the branched linker moiety is a polypeptide multivalent linker.

47. The multivalent protein conjugate of claim 46, wherein the polypeptide multivalent linker is selected from the group consisting of polylysines, polyornithines, polycysteines, polyglutamic acid and polyaspartic acid.

48. The multivalent protein conjugate of claim 46, wherein the polypeptide multivalent linker is a pennant or cascading polypeptide linker.

49. A method for treating a disease associated with abnormal angiogenesis, comprising:
administering to a subject with a disease associated with abnormal angiogenesis a multivalent protein conjugate of claim 1, 42, or 45.

50. The method of claim 49, wherein the disease associated with abnormal angiogenesis is a benign tumor or cancer.

51. The method of claim 50, wherein the benign tumor is selected from the group consisting of hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

52. The method of claim 50, wherein the cancer is selected from the group consisting of leukemia, breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, lymphomas, malignant melanomas, epidermoid carcinomas, and other carcinomas and sarcomas.

53. The method of claim 49, wherein the disease associated with abnormal angiogenesis is selected from the group consisting of restenosis, atherosclerosis, insults to body tissue due to surgery, abnormal wound healing, diseases that produce fibrosis of tissue, repetitive motion disorders, disorders of tissues that are not highly vascularized, and proliferative responses associated with organ transplants.


SEQUENCE LISTING (33 sequences)

SEQ ID NO: 1 - protein, 2 aa, Artificial sequence
  linker sequence
GG

SEQ ID NO: 2 - protein, 3 aa, Artificial sequence
  Linker sequence
GAG

SEQ ID NO: 3 - protein, 3 aa, Artificial sequence
  Linker sequence
GPA

SEQ ID NO: 4 - protein, 5 aa, Artificial sequence
  Linker sequence
GGGGS

SEQ ID NO: 5 - DNA, 35 bp, Artificial sequence
  PCR primer
atgaattcatggactctttagccagcttagttctc

SEQ ID NO: 6 - DNA, 29 bp, Artificial sequence
  PCR primer
atgtcgacgaggtccgctggtgcttgaga

SEQ ID NO: 7 - DNA, 33 bp, Artificial sequence
  PCR primer
ctagtcgacgagtccaaatcttgtgacaaaact

SEQ ID NO: 8 - DNA, 39 bp, Artificial sequence
  PCR primer
tccctgtctccgggtaaatgaggatccggtggtaccgat

SEQ ID NO: 9 - DNA, 35 bp, Artificial sequence
  PCR primer
ccaatcaaatccaagaagctagtcgacgagtccaa

SEQ ID NO: 10 - DNA, 39 bp, Artificial sequence
  PCR primer
ttgatcttgatcaatggcggtagacctttcgtagagatg

SEQ ID NO: 11 - DNA, 40 bp, Artificial sequence
  PCR primer
ggaattgatcaaaccgccggtttgtcgatgtgtgagatag

SEQ ID NO: 12 - DNA, 50 bp, Artificial sequence
  PCR primer
tggacttgatcttgatcaatggcgccggaagtgatacaggtagacctttc

SEQ ID NO: 13 - DNA, 49 bp, Artificial sequence
  PCR primer
gcattcatcactgtgaaacatggtgccggcttgatcaattccctacctc

SEQ ID NO: 14 - DNA, 2433 bp, Artificial sequence
  MVP-A
atggactctttagccagcttagttctctgtggagtcagcttgctcctttctggaactgtggaaggtgccatggacttgatcttgatcaatggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaacctccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagctagtcgacgagtccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 810 aa, Artificial sequence
  MVP-A
MDSLASLVLCGVSLLLSGTVEGAMDLILINGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLVDESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 2805 bp, Artificial sequence
  MVP-B
atggactctttagccagcttagttctctgtggagtcagcttgctcctttctggaactgtggaaggtgccatggacttgatcttgatcaatggcgccggaagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatggtgccggcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagctagtcgacgagtccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 17 - protein, 934 aa, Artificial sequence
  MVP-B
MDSLASLVLCGVSLLLSGTVEGAMDLILINGAGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHGAGLINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLVDESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 949 aa, Artificial sequence
  MVP-C
MDSLASLVLCGVSLLLSGTVEGAMSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHGAGGGGSGGGGSGGGGVEGAMDLILINLINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLVDESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 949 aa, Artificial sequence
  MVP-D
MDSLASLVLCGVSLLLSGTVEGAMDLILINLINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLVDESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGVEGAMSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHGA

SEQ ID NO: 20 - protein, 14 aa, Artificial sequence
  Linker
GGGGSGGGGSGGGG

SEQ ID NO: 21 - DNA, 31 bp, Artificial sequence
  Primer
tgttgacattgagctgggactagtagctttg

SEQ ID NO: 22 - DNA, 37 bp, Artificial sequence
  Primer
ccgtaattgattaagaatgacaactagtcagacaatg

SEQ ID NO: 23 - DNA, 35 bp, Artificial sequence
  Primer
gtttaggaattcgtcagccaccatggactctttag

SEQ ID NO: 24 - DNA, 20 bp, Artificial sequence
  Primer
tgagcatgaggcaggtgtac

SEQ ID NO: 25 - protein, 19 aa, Homo sapiens
MDSLASLVLCGVSLLLSGT

SEQ ID NO: 26 - protein, 95 aa, Homo sapiens
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQT

SEQ ID NO: 27 - protein, 210 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKH

SEQ ID NO: 28 - protein, 454 aa, Homo sapiens
LINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKL

SEQ ID NO: 29 - protein, 465 aa, Homo sapiens
VEGAMDLILINLINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKL

SEQ ID NO: 30 - protein, 484 aa, Homo sapiens
MDSLASLVLCGVSLLLSGTVEGAMDLILINLINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKL

SEQ ID NO: 31 - protein, 233 aa, Homo sapiens
DESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 24 aa, Homo sapiens
MDSLASLVLCGVSLLLSGTVEGAM

SEQ ID NO: 33 - protein, 30 aa, Homo sapiens
MDSLASLVLCGVSLLLSGTVEGAMDLILIN